SEQUENCE LISTING (3 sequences)

SEQ ID NO: 1 - DNA, 2520 bp, Homo sapiens
atgaagaaatggagcagcacagacttgggggcagctgcggacccactccaaaaggacacctgcccagaccccctggatggagaccctaactccaggccacctccagccaagccccagctctccacggccaagagccgcacccggctctttgggaagggtgactcggaggaggctttcccggtggattgccctcacgaggaaggtgagctggactcctgcccgaccatcacagtcagccctgttatcaccatccagaggccaggagacggccccaccggtgccaggctgctgtcccaggactctgtcgccgccagcaccgagaagaccctcaggctctatgatcgcaggagtatctttgaagccgttgctcagaataactgccaggatctggagagcctgctgctcttcctgcagaagagcaagaagcacctcacagacaacgagttcaaagaccctgagacagggaagacctgtctgctgaaagccatgctcaacctgcacgacggacagaacaccaccatccccctgctcctggagatcgcgcggcaaacggacagcctgaaggagcttgtcaacgccagctacacggacagctactacaagggccagacagcactgcacatcgccatcgagagacgcaacatggccctggtgaccctcctggtggagaacggagcagacgtccaggctgcggcccatggggacttctttaagaaaaccaaagggcggcctggattctacttcggtgaactgcccctgtccctggccgcgtgcaccaaccagctgggcatcgtgaagttcctgctgcagaactcctggcagacggccgacatcagcgccagggactcggtgggcaacacggtgctgcacgccctggtggaggtggccgacaacacggccgacaacacgaagtttgtgacgagcatgtacaatgagattctgatcctgggggccaaactgcacccgacgctgaagctggaggagctcaccaacaagaagggaatgatgccgctggctctggcagctgggaccgggaagatcggggtcttggcctatattctccagcgggagatccaggagcccgagtgcaggcacctgtccaggaagttcaccgagtgggcctacgggcccgtgcactcctcgctgtacgacctgtcctgcatcgacacctgcgagaagaactcggtgctggaggtgatcgcctacagcagcagcgagacccctaatcgccacgacatgctcttggtggagccgctgaaccgactcctgcaggacaagtgggacagattcgtcaagcgcatcttctacttcaacttcctggtctactgcctgtacatgatcatcttcaccatggctgcctactacaggcccgtggatggcttgcctccctttaagatggaaaaaactggagactatttccgagttactggagagatcctgtctgtgttaggaggagtctacttctttttccgagggattcagtatttcctgcagaggcggccgtcgatgaagaccctgtttgtggacagctacagtgagatgcttttctttctgcagtcactgttcatgctggccaccgtggtgctgtacttcagccacctcaaggagtatgtggcttccatggtattctccctggccttgggctggaccaacatgctctactacacccgcggtttccagcagatgggcatctatgccgtcatgatagagaagatgatcctgagagacctgtgccgtttcatgtttgtctacatcgtcttcttgttcgggttttccacagcggtggtgacgctgattgaagacgggaagaatgactccctgccgtctgagtccacgtcgcacaggtggcgggggcctgcctgcaggccccccgatagctcctacaacagcctgtactccacctgcctggagctgttcaagttcaccatcggcatgggcgacctggagttcactgagaactatgacttcaaggctgtcttcatcatcctgctgctggcctatgtaattctcacctacatcctcctgctcaacatgctcatcgccctcatgggtgagactgtcaacaagatcgcacaggagagcaagaacatctggaagctgcagagagccatcaccatcctggacacggagaagagcttccttaagtgcatgaggaaggccttccgctcaggcaagctgctgcaggtggggtacacacctgatggcaaggacgactaccggtggtgcttcagggtggacgaggtgaactggaccacctggaacaccaacgtgggcatcatcaacgaagacccgggcaactgtgagggcgtcaagcgcaccctgagcttctccctgcggtcaagcagagtttcaggcagacactggaagaactttgccctggtcccccttttaagagaggcaagtgctcgagataggcagtctgctcagcccgaggaagtttatctgcgacagttttcagggtctctgaagccagaggacgctgaggtcttcaagagtcctgccgcttccggggagaagtga

SEQ ID NO: 2 - DNA, 3360 bp, Homo sapiens
atgaagtgcagcctgaggaagatgtggcgccctggagaaaagaaggagccccagggcgttgtctatgaggatgtgccggacgacacggaggatttcaaggaatcgcttaaggtggtttttgaaggaagtgcatatggattacaaaactttaataagcaaaagaaattaaaaacatgtgacgatatggacaccttcttcttgcattatgctgcagcagaaggccaaattgagctaatggagaagatcaccagagattcctctttggaagtgctgcatgaaatggatgattatggaaatacccctctgcattgtgctgtagaaaaaaaccaaattgaaagcgttaagtttcttctcagcagaggagcaaacccaaacctccgaaacttcaacatgatggctcctctccacatagctgtgcagggcatgaataatgaggtgatgaaggtcttgcttgagcatagaactattgatgttaatttggaaggagaaaatggaaacacagctgtgatcattgcgtgcaccacaaataatagcgaagcattgcagattttgcttaacaaaggagctaagccatgtaaatcaaataaatggggatgtttccctattcaccaagctgcattttcaggttccaaagaatgcatggaaataatactaaggtttggtgaagagcatgggtacagtagacagttgcacattaactttatgaataatgggaaagccacccctctccacctggctgtgcaaaatggtgacttggaaatgatcaaaatgtgcctggacaatggtgcacaaatagacccagtggagaagggaaggtgcacagccattcattttgctgccacccagggagccactgagattgttaaactgatgatatcgtcctattctggtagcgtggatattgttaacacaaccgatggatgtcatgagaccatgcttcacagagcttcattgtttgatcaccatgagctagcagactatttaatttcagtgggagcagatattaataagatcgattctgaaggacgctctccacttatattagcaactgcttctgcatcttggaatattgtaaatttgctactctctaaaggtgcccaagtagacataaaagataattttggacgtaattttctgcatttaactgtacagcaaccttatggattaaaaaatctgcgacctgaatttatgcagatgcaacagatcaaagagctggtaatggatgaagacaacgatgggtgtactcctctacattatgcatgtagacaggggggccctggttctgtaaataacctacttggctttaatgtgtccattcattccaaaagcaaagataagaaatcacctctgcattttgcagccagttatgggcgtatcaatacctgtcagaggctcctacaagacataagtgatacgaggcttctgaatgaaggtgaccttcatggaatgactcctctccatctggcagcaaagaatggacatgataaagtagttcagcttcttctgaaaaaaggtgcattgtttctcagtgaccacaatggctggacagctttgcatcatgcgtccatgggcgggtacactcagaccatgaaggtcattcttgatactaatttgaagtgcacagatcgcttggatgaagacgggaacactgcacttcactttgctgcaagggaaggccacgccaaagccgttgcgcttcttctgagccacaatgctgacatagtcctgaacaagcagcaggcctcctttttgcaccttgcacttcacaataagaggaaggaggttgttcttacgatcatcaggagcaaaagatgggatgaatgtcttaagattttcagtcataattctccaggcaataaatgtccaattacagaaatgatagaatacctccctgaatgcatgaaggtacttttagatttctgcatgttgcattccacagaagacaagtcctgccgagactattatatcgagtataatttcaaatatcttcaatgtccattagaattcaccaaaaaaacacctacacaggatgttatatatgaaccgcttacagccctcaacgcaatggtacaaaataaccgcatagagcttctcaatcatcctgtgtgtaaagaatatttactcatgaaatggttggcttatggatttagagctcatatgatgaatttaggatcttactgtcttggtctcatacctatgaccattctcgttgtcaatataaaaccaggaatggctttcaactcaactggcatcatcaatgaaactagtgatcattcagaaatactagataccacgaattcatatctaataaaaacttgtatgattttagtgtttttatcaagtatatttgggtattgcaaagaagcggggcaaattttccaacagaaaaggaattattttatggatataagcaatgttcttgaatggattatctacacgacgggcatcatttttgtgctgcccttgtttgttgaaataccagctcatctgcagtggcaatgtggagcaattgctgtttacttctattggatgaatttcttattgtatcttcaaagatttgaaaattgtggaatttttattgttatgttggaggtaattttgaaaactttgttgaggtctacagttgtatttatcttccttcttctggcttttggactcagcttttacatcctcctgaatttacaggatcccttcagctctccattgctttctataatccagaccttcagcatgatgctaggagatatcaattatcgagagtccttcctagaaccatatctgagaaatgaattggcacatccagttctgtcctttgcacaacttgtttccttcacaatatttgtcccaattgtcctcatgaatttacttattggtttggcagttggcgacattgctgaggtccagaaacatgcatcattgaagaggatagctatgcaggtggaacttcataccagcttagagaagaagctgccactttggtttctacgcaaagtggatcagaaatccaccatcgtgtatcccaacaaacccagatctggtgggatgttattccatatattctgttttttattttgcactggggaaataagacaagaaataccaaatgctgataaatctttagaaatggaaatattaaagcagaaataccggctgaaggatcttacttttctcctggaaaaacagcatgagctcattaaactgatcattcagaagatggagatcatctctgagacagaggatgatgatagccattgttcttttcaagacaggtttaagaaagagcagatggaacaaaggaatagcagatggaatactgtgttgagagcagtcaaggcaaaaacacaccatcttgagccttag

SEQ ID NO: 3 - DNA, 3315 bp, Homo sapiens
atgtccttcgagggagccaggctcagcatgaggagccgcagaaatggtactatgggcagcacccggaccctgtactccagtgtatctcggagcacagacgtgtcctacagtgacagtgatttggtgaattttattcaggcaaattttaaaaaacgagaatgtgtcttctttaccagagactccaaggccatggagaacatatgcaagtgtggttatgcccagagccagcacatcgaaggcacccagatcaaccaaaatgagaagtggaactacaaaaaacataccaaggagtttccaacagacgccttcggggacattcagtttgagactctggggaagaaaggcaagtacttacgcttgtcctgtgacaccgactctgaaactctctacgaactgctgacccagcactggcacctcaaaacacccaacctggtcatttcagtgacgggtggagccaaaaactttgctttgaagccacgcatgcgcaagatcttcagcaggctgatttacatcgcacagtctaaaggtgcgtggattctcactggaggcactcactacggcctgatgaagtacataggcgaggtggtgagagacaacaccatcagcaggaactcagaagagaacatcgtggccattggcatcgcagcatggggcatggtctccaacagggacaccctcatcaggagctgtgatgatgagggacatttttcagctcaatacatcatggatgactttaccagagaccctctatacatcctggacaacaaccatacccacctgctgcttgtggacaacggttgtcatggacaccccacagtggaagccaagctccggaatcagctggaaaagtacatctctgagcgcaccagtcaagattccaactatggtggtaagatccccatcgtgtgttttgcccaaggaggtggaagagagactctaaaagccatcaacacctctgtcaaaagcaagatcccttgtgtggtggtggaaggctcggggcagattgctgatgtgatcgccagcctggtggaggtggaggatgttttaacctcttccatggtcaaagagaagctggtacgctttttaccacgcactgtgtcccggctgcctgaagaggaaattgagagctggatcaaatggctcaaagaaattcttgagagttctcacctactcacagtaattaagatggaagaggctggagatgagattgtgagcaacgccatttcctatgcgctgtacaaagccttcagcactaatgagcaagacaaggacaactggaatggacagctgaagcttctgctggagtggaaccagttggaccttgccagtgatgagatcttcaccaatgatcgccgctgggagtctgccgaccttcaggaggtcatgttcacggctctcataaaggacagacccaagtttgtccgcctctttctggagaatggcctgaatctgcagaagtttctcaccaatgaagtcctcacagagctcttctccacccacttcagcaccctagtgtaccggaatctgcagatcgccaagaactcctacaatgacgcactcctcacctttgtctggaagttggtggcaaacttccgtcgaagcttctggaaagaggacagaagcagcagggaggacttggatgtggaactccatgatgcatctctcaccacccggcacccgctgcaagctctcttcatctgggccattcttcagaacaagaaggaactctccaaggtcatttgggagcagaccaaaggctgtactctggcagccttgggggccagcaagcttctgaagaccctggccaaagttaagaatgatatcaacgctgctggggaatcggaggaactggccaatgaatatgagacccgagcagtggagttgttcaccgagtgttacagcaatgatgaagacttggcagaacagctactggtctactcctgcgaagcctggggtgggagcaactgtctggagctggcagtggaggctacagatcagcatttcatcgctcagcctggggtccagaatttcctttctaagcaatggtatggagagatttcccgagacacgaagaactggaagattatcctgtgtctattcatcatccccttagtgggctgtggcctcgtatcatttaggaagaaacccattgacaagcacaagaagctgctgtggtactatgtggccttcttcacgtcgcccttcgtggtcttctcctggaacgtggtcttctacatcgccttcctcctgctgtttgcctatgtgctgctcatggacttccactcagtgccacacacccccgagctgatcctctacgccctggtcttcgtcctcttctgtgatgaagtgaggcagtggtacatgaacggagtgaattatttcaccgacctatggaacgttatggacaccctgggactcttctacttcatagcgggtattgtattccggctccactcttctaataaaagctcgttgtactctgggcgcgtcattttctgtctggattacattatattcacgctaaggctcatccacattttcaccgtcagcaggaacttgggacccaagattataatgctgcagcggatgctgatcgacgttttcttcttcctgttcctctttgctgtgtggatggtggcctttggcgtggccagacaggggatcctaaggcaaaatgaacagcgctggagatggatcttccgctctgtcatctatgagccctacctggccatgtttggccaggttcccagtgacgtggatagtaccacatatgacttctcccactgtaccttctcgggaaatgagtccaagccactgtgtgtggagctggatgagcacaacctgccccgcttccctgagtggatcaccattccgctggtgtgcatctacatgctctccaccaatatccttctggtcaacctcctggtcgccatgtttggctacacggtaggcattgtacaggagaacaacgaccaggtctggaaattccagcggtacttcctggtgcaggagtactgcaaccgcctaaacatccccttccccttcgttgtcttcgcttatttctacatggtggtgaagaagtgtttcaaatgctgctgtaaagagaagaatatggagtctaatgcctgctgtttcagaaatgaggacaatgagactttggcgtgggagggtgtcatgaaggagaattaccttgtcaagatcaacacgaaagccaacgacaactcagaggagatgaggcatcggtttagacaactggactcaaagcttaacgacctcaaaagtcttctgaaagagattgctaataacatcaagtaa

(19) United States
(12) Patent Application Publication
(10) Pub. No.: US 2019/0330141 A1
(43) Pub. Date: Oct. 31, 2019

(54) Compositions With A Cooling Effect

(71) Applicant: The Procter & Gamble Company, Cincinnati, OH (US)

(72) Inventors: Gregory Mark Bunke, Lawrenceburg, IN (US); Heath A. Frederick, Harrison, OH (US); John Christian Haught, West Chester, OH (US); John August Wos, Mason, OH (US); Kenneth Edward Yelm, Hamilton, OH (US)

(21) Appl. No.: 16/398,304

(22) Filed: Apr. 30, 2019

Related U.S. Application Data
(60) Provisional application No. 62/664,428, filed on Apr. 30, 2018.

(51) Int. Cl. C07C 237/34; C07C 237/20; A61Q 11/00; A61P 3/04; A61P 3/10; A61P 1/00; A61P 25/00

(52) U.S. Cl. CPC C07C 237/34; C07C 237/20; A61Q 11/00; A61P 25/00; A61P 3/10; A61P 1/00; A61P 3/04

(57) ABSTRACT

Personal care compositions, such as oral care and skin care compositions comprising one or more coolants. The pleasant cool sensation provided by a coolant is enhanced in terms of quicker onset, greater intensity, impact or longer duration, which improves appeal and acceptability of the compositions to consumers. Also, a treatment for excess adipose tissue by applying an activating compound directly to a targeted area.

### FIELD OF THE INVENTION

The present invention relates to compounds that are useful as sensates. The present invention relates to aromatic, adamantyl, and highly branched aliphatic compounds that can be used as coolants. Also, the present invention relates to personal care compositions, such as oral care compositions, containing a flavor/perfume system comprising compositions with a cooling effect.

### BACKGROUND OF THE INVENTION

Oral care products, such as dentifrice and mouthwash, are routinely used by consumers as part of their oral care hygiene regimens. It is well known that oral care products can provide both therapeutic and cosmetic hygiene benefits to consumers. Therapeutic benefits include caries prevention which is typically delivered through the use of various fluoride salts; gingivitis prevention, by the use of an antimicrobial agent such as stannous fluoride, triclosan, essential oils; or hypersensitivity control through the use of ingredients such as strontium chloride or potassium nitrate. Cosmetic benefits provided by oral care products include the control of plaque and calculus formation, removal and prevention of tooth stain, tooth whitening, breath freshening, and overall improvements in mouth feel impression, which can be broadly characterized as mouth feel aesthetics. Calculus and plaque along with behavioral and environmental factors lead to formation of dental stains, significantly affecting the aesthetic appearance of teeth. Behavioral and environmental factors that contribute to teeth staining propensity include regular use of coffee, tea, cola or tobacco products, and also the use of certain oral products containing ingredients that promote staining, such as cationic antimicrobials and metal salts.

Thus, daily oral care at home requires products with multiple ingredients working by different mechanisms to provide the complete range of therapeutic and aesthetic benefits, including anticaries, antimicrobial, antigingivitis, antiplaque, anticalculus and anti-erosion, as well as antiodor, mouth refreshment, stain removal, stain control and tooth whitening. In order for daily use oral care products, such as dentifrice and rinses to provide complete oral care it is often necessary to combine actives and additives, many of which have the disadvantage of causing negative aesthetics during use, in particular unpleasant taste and sensations and stain promotion. The unpleasant taste and mouth sensations have been described as having one or more of bitter, metallic, astringent, salty, numbing, stinging, burning, or prickling, and even irritating aspects. Typical ingredients for oral care use that are associated with these aesthetic negatives include antimicrobial agents such as cetyl pyridinium chloride, chlorhexidine, stannous and zinc salts; tooth bleaching agents such as peroxides; antitartar agents such as pyrophosphate, tripolyphosphate and hexametaphosphate; and excipients such as baking soda and surfactants. To mitigate the aesthetic negatives from these ingredients, oral care products are typically formulated with flavoring agents, sweeteners and coolants to taste as good as possible and provide a pleasant experience. In particular, it is desirable for oral care products to provide a refreshing cooling sensation during and after use. In addition to mitigation of negative sensations, sensate molecules are formulated into oral care compositions to convey a signal of efficacy. Such signals of efficacy include cooling, tingling, numbing, warming, sweetness, and rheological sensations such as phase change and fizzing or bubbling.

A large number of coolant compounds of natural or synthetic origin have been described. The most well-known compound is menthol, particularly 1-menthol, which is found naturally in peppermint oil, notably of Mentha arvensis L and Mentha viridis L. Of the menthol isomers, the 1-isomer occurs most widely in nature, and is typically associated with the name menthol having coolant properties. L-menthol has the characteristic peppermint odor, a clean fresh taste, and exerts a cooling sensation when applied to the skin and mucosal surfaces. Other isomers of menthol (neomenthol, isomenthol and neoisomenthol) have somewhat similar, but not identical odor and taste, i.e., some having disagreeable notes described as earthy, camphor, musty. The principal difference among the isomers is in their cooling potency. L-menthol provides the most potent cooling, i.e., having the lowest cooling threshold of about 800 ppb, i.e., the concentration where the cooling effect could be clearly recognized. At this level, there is no cooling effect for the other isomers. For example, d-neomenthol is reported to have a cooling threshold of about 25,000 ppb and 1-neomenthol about 3,000 ppb.

Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds, such as N-ethyl-ρ-menthan-3-carboxamide, known commercially as “WS-3”, and others in the series, such as WS-5 (N-ethoxycarbonylmethyl-ρ-menthan-3-carboxamide), WS-12 [N-(4-methoxyphenyl)-ρ-menthan-3-carboxamide] and WS-14 (N-tert-butyl-ρ-menthan-3-carboxamide). Examples of menthane carboxy esters include WS-4 and WS-30. An example of a synthetic carboxamide coolant that is structurally unrelated to menthol is N,2,3-trimethyl-2-isopropylbutanamide, known as “WS-23”.

However, the cyclohexane moiety can be expensive to incorporate into personal care compositions and oral care compositions. Thus, an object of the present invention is to replace the cyclohexane moiety with an aromatic, adamantyl, or highly branched alkyl moiety to reduce costs while maintaining and/or improving the cooling performance typically provided by menthol or derivatives previously synthesized.

The present invention provides compositions comprising one or more coolants, wherein the cooling and refreshing sensation provided by the coolant(s) is potentiated in terms of onset, intensity, and/or duration.

Additionally, the present invention provides methods and medical devices for the local activation of adipocytes by applying an activating compound. The activating compound activates thermogenesis in white, brown, or beige adipose tissue, which can lead to the generation of heat, lipolysis of adipose tissue, and ultimately lead to the overall reduction in quantity and size of adipose tissue.

### SUMMARY OF THE INVENTION

A compound comprising the following structure or salts thereof:

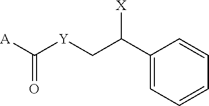

A is phenyl, pyridinyl, adamantyl, B, alkylsubstituted phenyl, alkylsubstituted pyridinyl, —O-phenyl, —O-pyridinyl, —O-adamantyl, —O—B, —O-(alkylsubstituted phenyl), —O-(alkylsubstituted pyridinyl), —N— phenyl, —N-pyridinyl, —N-adamantyl, —N—B, —N-(alkylsubstituted phenyl), or-N-(alkylsubstituted pyridinyl);
Y is —O—, —NH—, or nil in the case wherein A connects to the carbonyl functional group with an oxygen or nitrogen atom;

### X is —OH, —O-AA, —NH₂, or —NH-AA;

B is tert-butyl, isopropyl, —C(isopropyl)₂(CH₃), or —(CH)═C(CH₃)—CH₂—CH₂—(CH)═C(CH₃)₂; and
AA is an amino acid.

A compound is provided having the structure shown above, wherein the compound at a concentration of about 5.2E-5% provides a greater activation of TRPM8 than WS5 at a concentration of about 30 mM; a greater activation of TRPA1 than allyl isothiocyanate at a concentration of about 50 mM; and a greater activation of TRPV1 than capsaicin at a concentration of about 350 nM.

A compound having the structure shown above is provided, wherein the compound at a concentration of about 5.2E-5% provides at least about 100%, 105%, 110%, 115%, 120% 125% or 130% activation of TRPM8 when compared to WS5 at a concentration of about 30 mM; at least about 100%, 130%, 140%, 150%, 160%, 170%, 180%, 190%, 200%, 210%, 220%, 230% or 240% activation of TRPA1 when compared to allyl isothiocyanate at a concentration of about 50 mM; and at least about 95%, 100%, 105%, 110%, or 115% activation of TRPV1 when compared to capsaicin at a concentration of about 350 nM.

A method of promoting thermogenesis comprising contacting one or more adipocytes with an activating compound, wherein the activating compound comprises the following structure or salts thereof:

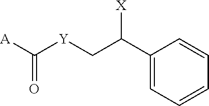

A is phenyl, pyridinyl, adamantyl, B, alkylsubstituted phenyl, alkylsubstituted pyridinyl, —O-phenyl, —O-pyridinyl, —O-adamantyl, —O—B, —O-(alkylsubstituted phenyl), —O-(alkylsubstituted pyridinyl), —N— phenyl, —N-pyridinyl, —N-adamantyl, —N—B, —N-(alkylsubstituted phenyl), or-N-(alkylsubstituted pyridinyl);
Y is —O—, —NH—, or nil in the case wherein A connects to the carbonyl functional group with an oxygen or nitrogen atom;

### X is —OH, —O-AA, —NH₂, or —NH-AA;

B is tert-butyl, isopropyl, —C(isopropyl)₂(CH₃), or —(CH)═C(CH₃)—CH₂—CH₂—(CH)═C(CH₃)₂; and
AA is an amino acid.

A personal care composition comprising an activating compound with the following structure or salts thereof:

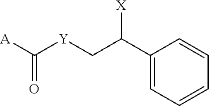

A is phenyl, pyridinyl, adamantyl, B, alkylsubstituted phenyl, alkylsubstituted pyridinyl, —O-phenyl, —O-pyridinyl, —O-adamantyl, —O—B, —O-(alkylsubstituted phenyl), —O-(alkylsubstituted pyridinyl), —N— phenyl, —N-pyridinyl, —N-adamantyl, —N—B, —N-(alkylsubstituted phenyl), or-N-(alkylsubstituted pyridinyl);
Y is —O—, —NH—, or nil in the case wherein A connects to the carbonyl functional group with an oxygen or nitrogen atom;

### X is —OH, —O-AA, —NH₂, or —NH-AA;

B is tert-butyl, isopropyl, —C(isopropyl)₂(CH₃), or —(CH)═C(CH₃)—CH₂—CH₂—(CH)═C(CH₃)₂; and
AA is an amino acid.

A method of activating TRPM8 comprising contacting the composition of the previous compositions with an oral cavity.

### DETAILED DESCRIPTION OF THE INVENTION

The present invention is directed to the discovery that certain compounds deliver the means to drive a cooling response at low concentrations. A second object of this invention shows the discovery that certain compounds activate the transient receptor potential cation channel subfamily M member 8 (TRPM8) receptor, known as the menthol receptor, which drives the non-thermal cooling perception. As activators of TRPM8, these compounds can also be used to promote thermogenesis, which can lead to adipocyte differentiation from adipocyte precursors and/or the conversion of white adipocytes to beige and/or brown adipocytes.

The present invention is thus based on the discovery that select molecules can be used to drive a cooling response when formulated into consumer products. This invention also shows the discovery that select compounds can provide long lasting cooling at very low levels, allowing for formulation efficiencies, in particular coolant compounds (coolants), such as described below.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

The foregoing summary is not intended to define every aspect of the invention, and additional aspects are described in other sections, such as the Detailed Description. In addition, the invention includes, as an additional aspect, all embodiments of the invention narrower in scope in any way than the variations defined by specific paragraphs set forth herein. For example, certain aspects of the invention that are described as a genus, and it should be understood that every member of a genus is, individually, an aspect of the invention. Also, aspects described as a genus or selecting a member of a genus should be understood to embrace combinations of two or more members of the genus. With respect to aspects of the invention described or claimed with “a” or “an,” it should be understood that these terms mean “one or more” unless context unambiguously requires a more restricted meaning. The term “or” should be understood to encompass items in the alternative or together, unless context unambiguously requires otherwise. If aspects of the invention are described as “comprising” a feature, embodiments also are contemplated “consisting of” or “consisting essentially of” the feature.

Features of the compositions and methods are described below. Section headings are for convenience of reading and not intended to be limiting per se. The entire document is intended to be related as a unified disclosure, and it should be understood that all combinations of features described herein are contemplated, even if the combination of features are not found together in the same sentence, or paragraph, or section of this document. It will be understood that any feature of the methods or compounds described herein can be deleted, combined with, or substituted for, in whole or part, any other feature described herein.

All measurements referred to herein are made at 25° C. unless otherwise specified.

By “personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs, such as Vicks Vaporub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats.

The present invention is also directed towards “oral care compositions” as used herein refers to compositions in a form that is deliverable to a mammal in need via the oral cavity, mouth, throat, nasal passage or combinations thereof. Nonlimiting examples include liquid compositions, cough syrups, respiratory preparations, beverage, supplemental water, pills, soft gels, tablets, capsules, gel compositions, foam compositions, saline wash and combinations thereof. Liquid compositions, gel compositions can be in a form that is directly deliverable to the mouth and throat. These compositions and/or preparations can be delivered by a delivery device selected from droppers, pump, sprayers, liquid dropper, saline wash delivered via nasal passageway, cup, bottle, liquid filled gel, liquid filled gummy, center filled gum, chews, films, center filled lozenge, gum filled lozenge, pressurized sprayers, atomizers, air inhalation devices, liquid filled compressed tablet, liquid filled gelatin capsule, liquid filled capsule, squeezable sachets, power shots, and other packaging and equipment, and combinations thereof. The sprayer, atomizer, and air inhalation devices can be associated with a battery or electric power source.

The present invention is also directed towards a respiratory preparation. In one embodiment the respiratory preparation comprises a film forming agent; and a thickening agent. The preparation provides on demand relief. The preparation can work to physically coat the mouth and throat creating a soothing barrier over the epithelial cells that line the throat layer. The preparation can additionally, reduce inflammation and relieve minor pain associated with a cough and/or sore throat. Preferably the respiratory preparation would not contain a pharmaceutical active.

The present invention is also directed to lotion compositions and to absorbent articles, particularly disposable absorbent articles, having a lotion treatment composition applied thereon. Disposable absorbent articles can be baby diapers or feminine hygiene articles, including incontinence devices and catamenial products, such as tampons, sanitary napkins, pantiliners, interlabial products, and the like. For convenience, the invention is disclosed below with respect to the embodiment of a catamenial device, such as a sanitary napkin or pantiliner.

The absorbent article can comprise any known or otherwise effective topsheet, such as one which is compliant, soft feeling, and non-irritating to the body of the wearer. Suitable topsheet materials include a liquid pervious material that is oriented towards and contacts the body of the wearer, thereby permitting body discharges to rapidly penetrate through the topsheet without allowing fluid to flow back through the topsheet to the skin of the wearer. The topsheet, while capable of allowing rapid transfer of fluid through it, also provides for the transfer or migration of the lotion composition onto an external or internal portion of a body of the wearer. A suitable topsheet can be made of various materials, such as woven and nonwoven materials; apertured film materials including apertured formed thermoplastic films, apertured plastic films, and fiber-entangled apertured films; hydro-formed thermoplastic films; porous foams; reticulated foams; reticulated thermoplastic films; thermoplastic scrims; or combinations thereof, as is well known in the art of making catamenial products such as sanitary napkins, pantiliners, incontinence pads, and the like.

A lotion composition of the present invention comprises at least one rheology structurant, which typically is a solid. The lotion composition can further comprise other optional ingredients, like surface energy modifiers. In one embodiment, a lotion composition consists essentially of, or consists of, a rheology structurant, such as a microcrystalline wax, alkyl dimethicone, ethylene glycol dibehenate, ethylene glycol distearate, glycerol tribehenate, glycerol tristearate, and ethylene bis leamide. A present lotion composition can contain a single rheology structurant or a mixture of two or more rheology structurants.

In preparing a lotioned catamenial device according to the present invention, the lotion composition can be applied to the outer surface of the absorbent article, such as, for example, the outer surface of the topsheet. Any of a variety of application methods that distribute lubricious materials having a molten or liquid consistency can be used, such as, for example, as set forth in U.S. Pat. No. 5,968,025 and U.S. Pub. App. No. 2005/0208113. Suitable methods include but are not limited to spraying, printing (e.g., flexographic printing), coating (e.g., gravure coating), extrusion, dipping, or combinations of these application techniques, e.g., spraying the lotion composition on a rotating surface, such as a calender roll, that then transfers the composition to the outer surface of the sanitary napkin topsheet. Additionally, the manner of applying the lotion composition to a portion of a catamenial device can be such that the substrate or component does not become saturated with the lotion composition. The lotion composition can be applied to the catamenial device at any point during assembly. For example, the lotion composition can also be applied to the outer surface of the topsheet before it is combined with the other raw materials to form a finished catamenial device.

The term “dentifrice”, as used herein, includes tooth or subgingival-paste, gel, or liquid formulations unless otherwise specified. The dentifrice composition may be a single-phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

The term “orally acceptable carrier or excipients” includes safe and effective materials and conventional additives used in oral care compositions including but not limited to fluoride ion sources, anti-calculus or anti-tartar agents, buffers, abrasives such as silica, alkali metal bicarbonate salts, thickening materials, humectants, H₂O, surfactants, titanium dioxide, flavorants, sweetening agents, xylitol, coloring agents, and mixtures thereof.

Herein, the terms “tartar” and “calculus” are used interchangeably and refer to mineralized dental plaque biofilms.

The term “adipocyte”, as used herein, refers to a cell primarily composing adipose tissue, which specializes in storing energy as fat or triglycerides.

The term “white adipocyte”, as used herein, refers to an adipocyte whose main function is to act as a reservoir of triglycerides or fat for future energy utilization.

The term “brown adipocyte”, as used herein, refers to an adipocyte whose main function is to convert excess energy into body heat using non-shivering thermogenesis. Brown adipocytes are characterized by having a high proportion of mitochondria.

The term “beige adipocyte”, as used herein, refers to a white-like adipocyte that can induce non-shivering thermogenesis.

| SEQ ID NO | Sequence |
|---|---|
| 1 | Human TRPV1 DNA sequence |
| 2 | Human TRPA1 DNA sequence |
| 3 | Human TRPM8 DNA sequence |

A sequence listing that sets forth the nucleotide sequences for SEQ ID NO 1-3 herein is being filed concurrently with the present application as an ASCII text file titled “15171_Nucleotide_Sequence_Listing.” The ASCII text file was created on 26 Mar. 2018 and is 13 Kbytes in size. In accordance with MPEP § 605.08 and 37 CFR § 1.52(e), the subject matter in the ASCII text file is incorporated herein by reference.

The term “TRPV1” or “TRPV1 receptor”, as used herein, refers to the transient receptor potential vanilloid receptor 1, which is a ligand-gated, non-selective cation channel preferentially expressed on small-diameter sensory neurons and detects noxious as well as other substances. The TRPV1 receptor is provided as SEQ ID NO: 1. The TRPV1 receptor responds to, for example, both noxious and painful stimuli. A noxious stimulus would include those that give a burning (i.e. hot) sensation.

The term “TRPV1 agonist”, as used herein, refers to any compound, which at a concentration of 1 mM gives a calcium flux count of at least 1000 counts or 20% above the background level of calcium present in the cell according to the FLIPR method, as discussed herein. The term “count” is defined as the change in fluorescence of the cell lines due to the influx of calcium across the cell membrane, which reacts with the calcium sensitive dye present within the cells.

The term “TRPV1 antagonist”, as used herein, refers to any compound which at a concentration of 1 mM gives a reduction in calcium flux count of at least 1000 counts or 20% below the activation of TRPV1 receptor by 350 μM capsaicin.

The term “TRPV1 desensitizer”, as used herein, refers to any compound, which shows agonist activity and causes a decrease in activation by a known TRPV1 agonist.

The term “TRPA1” or “TRPA1 receptor”, as used herein, refers to the transient receptor potential cation channel, subfamily A, member 1, having a large cysteine-rich N-terminus that contains 18 predicted ankyrin repeats. The TRPA1 receptor is provided as SEQ ID NO: 2. TRPA1 is a ligand-gated, non-selective cation channel preferentially expressed on small diameter sensory neurons.

The term “TRPA1 agonist”, as used herein, refers to any compound, which at a concentration of 1 mM gives a calcium flux count of at least 1000 counts or 20% above the background level of calcium present in the cell according to the FLIPR method, as discussed herein. The term “count” is defined as the change in fluorescence of the cell lines due to the influx of calcium across the cell membrane, which reacts with the calcium sensitive dye present within the cells.

The term “TRPA1 antagonist”, as used herein, refers to any compound, which at a concentration of 1 mM gives a reduction in calcium flux count of at least 1000 counts or 20% below the activation of TRPA1 receptor by 50 mM allyl isothiocyanate.

The term “TRPA1 desensitizer”, as used herein, refers to any compound, which shows agonist activity and causes a decrease in activation by a known TRPA1 agonist.

The term “TRPM8” or “TRPM8 receptor”, as used herein, refers to cold- and menthol-sensitive receptor (CMR1) or TRPM8. The TRPM8 nomenclature for the receptor comes from its characterization as a non-selective cation channel of the transient receptor potential (TRP) family that is activated by stimuli including low temperatures, menthol and other chemical coolants. The TRPM8 receptor is provided as SEQ ID NO: 3.

The cooling receptor conventionally known as TRPM8, or the menthol receptor, has been demonstrated as a means to differentiate intensity and duration of organic molecules that initiate and propagate the non-thermal cooling perception (D.D.Mckemy, The Open Drug Discovery Journal 2:81-88 2010). McKemy reported the EC50 values of many agonists to TRPM8 which span the range of 100 nM to 19 mM, thus showing the channel can be activated across a wide range of structures at varying concentrations. This channel also has the nomenclature of CRM1 and TRPP8. The later was designated as such due to its identification with prostate cells, where it was employed as a means to identify molecules targeted towards prostate cancer.

The term “TRPM8 agonist”, as used herein, refers to any compound, which when added to a TRPM8 receptor, according to the FLIPR method, as discussed herein, produces any increase in fluorescence over background.

The term “TRPM8 antagonist”, as used herein, refers to any compound, which inhibits activation of the TRPM8 receptor by a known TRPM8 agonist. Using the FLIPR method, as discussed herein a molecule that has >20% reduction in calcium flux compared to the WS5 activated TRPM8 receptor is considered a TRPM8 antagonist.

The term “TRPM8 enhancer”, as used herein, refers to any compound that boosts the calcium flux activity of an agonist that directly activates TRPM8, but does not directly activate TRPM8.

The term potency, as defined by the Merck Manual, refers to the concentration (EC₅₀) or dose (ED₅₀) of a chemistry required to produce 50% of the chemistry's maximal effect as depicted by a graded dose-response curve. EC₅₀ equals Kd (Dissociation constant, which is a measure of 50% of the substance in question bound to the receptor) when there is a linear relationship between occupancy and response. Often, signal amplification occurs between receptor occupancy and response, which results in the EC₅₀ for response being much less (ie, positioned to the left on the abscissa of the log dose-response curve) than KD for receptor occupancy. Potency depends on both the affinity of chemistry for its receptor, and the efficiency with which chemistry-receptor interaction is coupled to response. The dose of chemistry required to produce an effect is inversely related to potency. In general, low potency is important only if it results in a need to administer the chemistry in large doses that are impractical. Quantal dose-response curves provide information on the potency of chemistry that is different from the information derived from graded dose-response curves. In a quantal dose-response relationship, the EDso is the dose at which 50% of individuals exhibit the specified quantal effect.

Coolants or compounds that have a physiological cooling effect particularly on oral and other mucosal surfaces and skin are common ingredients in a wide variety of products, including edible compositions, personal care compositions, and in flavor or perfume compositions. Examples of edible compositions include confectionery, candies, chocolate, chewing gum, beverages and oral medicines. Personal care compositions, including oral care compositions, have been described previously. The pleasant cooling sensation provided by coolants contributes to the appeal and acceptability of the products. In particular, oral care products, such as dentifrices and mouthwashes are formulated with coolants because they provide breath freshening effects and a clean, cool, fresh feeling in the mouth.

While not wishing to being bound by theory, disclosed herein are methods and devices capable of inducing thermogenesis in brown, beige, and white adipocytes. As described herein, non-shivering thermogenesis can be stimulated by cold temperatures. Activation of TRMP8 and/or promotion of thermogenesis in one or more adipocytes and/or adipose tissue can lead to adipocyte differentiation (i.e. pre-adipocytes preferentially developing into brown adipocytes instead of white adipocytes) and/or the conversion of white adipocytes to beige and/or brown adipocytes.

Without wishing to be bound by theory, the activating compounds disclosed herein can activate TRPM8 and/or promote thermogenesis in one or more adipocytes. The activation of TRPM8 can promote thermogenesis or thermogenesis can be directly promoted after contact between the activating compound and one or more adipocytes. The activation of TRPM8 and/or the promotion of thermogenesis can lead to preferential formation of beige and brown adipocytes over white adipocytes from preadipocyte cells. Additionally, the activation of TRPM8 and/or the promotion of thermogenesis can lead to the conversion of white adipocytes to beige and/or brown adipocytes. Additionally, the activation of TRPM8 and/or the promotion of thermogenesis can lead to increased mitochondrial activity in white adipocytes, which may make them act more like beige or brown adipocytes.

Components of the present compositions are described in the following paragraphs.

### Compositions

It is now well established that sensations such as cool or cold can be attributed to activation of receptors at peripheral nerve fibers by a stimulus, such as low temperature or a chemical coolant, which produces electrochemical signals that travel to the brain, which then interprets, organizes and integrates the incoming signals into a perception or sensation. Different classes of receptors have been implicated in sensing cold temperatures or chemical coolant stimuli at mammalian sensory nerve fibers. Among these receptors, a major candidate involved in sensing cold has been identified and designated as cold- and menthol-sensitive receptor (CMR1) or TRPM8. The TRPM8 nomenclature for the receptor comes from its characterization as a non-selective cation channel of the transient receptor potential (TRP) family, which is activated by stimuli including low temperatures, menthol and other chemical coolants. However, the precise mechanisms underlying the perception of a pleasant cooling sensation on skin or oral surfaces are presently not clearly understood. While it has been demonstrated that the TRPM8 receptor is activated by menthol and other coolants, it is not fully understood what other receptors may be involved, and to what extent these receptors need to be stimulated or perhaps suppressed in order for the overall perceived sensation to be pleasant, cooling and refreshing. For example, menthol is widely used as a cooling agent, but menthol can also produce other sensations including tingling, burning, prickling and stinging as well as a minty smell and bitter taste. Thus, it can be inferred that menthol acts on many different receptors, including cold, warm, pain and taste receptors.

Ideally, a coolant can produce a cooling or freshness sensation similar to that produced by menthol, but without certain of the disadvantages associated with menthol, such as flavor modification, bitter aftertaste, off-flavor, strong odor and burning or irritating sensation, particularly at high concentrations. It is desirable that the coolant compounds barely possess a distinctive odor or flavor while providing a pleasant fresh cool sensation of prolonged duration, in order that the effect can still be perceived for a considerable time after use, for example, longer than 15 minutes. Menthol generally provides an initial high cooling impact, but its effect is somewhat transient in that the cool sensation drops sharply within a few minutes after use. By contrast, a number of longer lasting coolant compounds may fail to provide an immediate cooling perception, i.e., within a few seconds of application, particularly when used at low levels. Thus, there is a continuing need for means to potentiate the activity of coolant chemicals, in terms of quickening the onset of the cooling sensation, intensifying the cooling sensation, especially at lower concentrations, and producing a longer lasting sensation of cooling and freshness than what menthol provides.

As stated previously, the present invention is directed to the discovery that certain compounds can deliver the means to drive a cooling response at low concentrations with lower formulation costs.

Suitable compounds can be represented by Formula I. Suitable compounds can also be salts of Formula I.

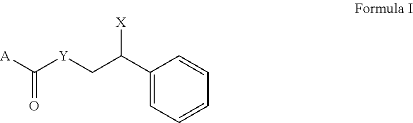

A is phenyl, pyridinyl, adamantyl, B, alkylsubstituted phenyl, alkylsubstituted pyridinyl, —O-phenyl, —O-pyridinyl, —O-adamantyl, —O—B, —O-(alkylsubstituted phenyl), —O-(alkylsubstituted pyridinyl), —N— phenyl, —N-pyridinyl, —N-adamantyl, —N—B, —N-(alkylsubstituted phenyl), or-N-(alkylsubstituted pyridinyl)
Y is —O—, —NH—, or nil in the case wherein A connects to the carbonyl functional group with an oxygen or nitrogen atom

### X is —OH, —O-AA, —NH₂, or —NH-AA

B is tert-butyl, isopropyl, —C(isopropyl)₂(CH₃), or —(CH)═C(CH₃)—CH₂—CH₂—(CH)═C(CH₃)₂
AA is an amino acid

Suitable compounds can also be represented by Formula II. Suitable compounds can also be salts of Formula II.

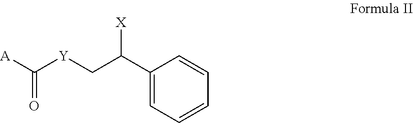

A is phenyl, adamantyl, B, alkylsubstituted phenyl, 2-isopropyl-5-methylphenyl, —O-phenyl, —O— adamantyl, —O—B, —O-(alkylsubstituted phenyl), or —O-(2-isopropyl-5-methylphenyl)
Y is —O—, —NH—, or nil in the case wherein A connects to the carbonyl functional group with an oxygen or nitrogen atom

### X is —OH, —O-AA, —NH₂, or —NH-AA

B is tert-butyl, isopropyl, or —C(isopropyl)₂(CH₃)
AA is alanine or glycine

Suitable compounds can be represented by Formula III. Suitable compounds can also be salts of Formula III.

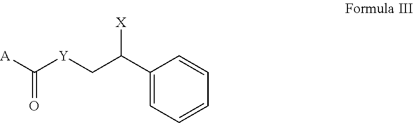

A is adamantyl, —C(isopropyl)₂(CH₃), 2-isopropyl-5-methylphenyl, or —O-(2-isopropyl-5-methylphenyl),
Y is —O—, —NH—, or nil in the case wherein A connects to the carbonyl functional group with an oxygen or nitrogen atom

### X is —O-AA, —NH₂, or —NH-AA

AA is alanine or glycine

Other suitable uses for long lasting TRPM8 activity, would be for food applications; skin conditions, such as treatments for non-keratinzed stratified epithelium; analgesic applications as pain mitigation agents; reductions in inflammation; additives to cigarettes; topical salves for muscle pain, for chronic pain from osteoarthritis, and for chemotherapy induced neuropathy; skin barrier recovery accelerants; and antipruritic or antiseptic medications; and for vasoconstriction in relaxed vessels.

One of the factors determining the levels of use for compounds of the present invention, depend upon the targeted TRPM8 area of the body. For example, in an oral application of a compound of the present invention, such as dentifrice, floss, chewing gum, or white strip, the levels of use may be from about 0.00001% to about 0.1%; from about 0.00005% to about 0.1%; from about 0.0001% to about 0.05%;

or from about 0.001% to about 0.01% by weight of the composition. When a compound of the present invention is used in a mouthwash, the level of use may be from about 0.000001% to about 0.01% or from about 0.0001% to about 0.001% by weight of the composition. When a compound of the present invention, is delivered topically, for example in shampoos and lotions the levels may be from about 0.001% to about 0.5% by weight of the composition or from about 0.01% to about 0.4% by weight of the composition.

When a compound of the present invention is combined with a TRPA1 agonist, TRPV1 agonist, or both, the level of use of a TRPA1 or TRPV1 agonist would be in the range of about 0.001% to about 0.5% or from about 0.01% to about 0.2% by weight of the composition of either the TRPA1 or TRPV1 agonists, where both TRPA1 agonists and/or TRPV1 agonists may be added separately or simultaneously to the compositions comprising activating compounds. When another TRPM8 agonist, in addition to a compound of the present invention, is used, the level of use of the additional TRPM8 agonist may be from about 0.001% to about 0.5% or from about 0.005% to about 0.3% by weight of the composition. If a TRPM8 enhancer is used, in addition to a compound of the present invention, it may be added in a range of from about 0.001% to about 0.2% or from about 0.005% to about 0.1% by weight of the composition. The compositions may contain multiple TRPA1 and TRPV1 agonists in the ranges stated.

Cooling can be further enhanced by combining with select TRPV1 warming agents. Non-limiting examples of TRPV1 warming agents would be capsaicin, vanillyl butyl ether, vanillyl ethyl ether, zingerone, and piperine. Other warming agents have previously been described in U.S. Pat. No. 6,673,844.

Combinations of a compound from Formula I-III with other TRPM8 coolants may provide a quicker onset of cooling with a higher intensity than either used alone. Combining a compound from Formula I-III with another coolant would allow for even less of a compound from Formula I-III to be used while still providing considerable (>3 hours) freshness longevity, which may be perceived as a cooling sensation. Examples of coolant combinations that could be used include WS23, menthane diols, menthyl carboxamide derivatives, such as WS3, WS5, N-(4-cyanomethylphenyl)-p-menthanecarboxamide, and WS12.

### Inducing Thermogenesis

The compounds described herein can also be used to induce thermogenesis in adipocytes. Adipocytes are complex cells that have multiple functions, depending on their physical location and physiological status, including storage of energy (fat), mechanical (fat pads, covering delicate organs such as eyes), and adaptive thermogenesis. Adipocytes play critical roles in systemic energy and metabolic regulation. Three forms of adipocytes, white, brown and beige have been described in humans.

White adipocytes store energy and serve as major secretory and endocrine organs that secrete adipokines (e.g. leptin, adiponectin, resistin), which perform various metabolic functions. White adipocytes make up the bulk of fatty tissues in animals. White adipose tissue is the most common type of adipose tissue and is characterized by a narrow rim of cytoplasm with its nucleus pressed near the margin of the cell surrounding a single large membrane-enclosed lipid droplet and a few mitochondria, modest blood supply, and serves as a depot of stored energy. Also, white adipocyte is an endocrine organ and secretes, leptin, adiponectin, and asprosin hormones that regulate various metabolic process. New adipocytes in white adipose tissue are formed throughout life from a pool of precursor cells. These are needed to replace those that die (after an average life span of 10 years). In addition to serving as a major source of energy reserves, white adipose tissue also provides some mechanical protection and insulation to the body. Obesity is the excessive accumulation of white adipose tissue.

Brown adipocytes are highly specialized cells that dissipate stored chemical energy in the form of heat. They achieve this by uncoupling protein-1 (UCP-1), a mitochondrial protein that is present in brown adipose tissue. Cold stimuli and/or certain molecules can activate UCP-1 in the existing brown adipocytes, thus increasing total energy expenditure by a magnitude proportional to the number of available brown adipocytes. Adult humans have significant depots of brown adipose tissue, and these can be activated when exposed to cold temperatures. Brown adipose tissue is a key site of heat production (thermogenesis). Brown adipose tissue is characterized by the presence of cytoplasm throughout the cell with a central nucleus, many small lipid droplets, many mitochondria that are rich in UCP-1, and rich in blood supply. UCP-1, when activated, short circuits the electrochemical gradient that drives ATP synthesis to generate heat instead. Brown adipose tissue provides a vital source of heat to maintain body temperature. Brown adipose tissue is activated when the body temperature drops.

Beige adipocytes are cells that form from white adipocytes upon stimulation. Beige adipocytes can be found interspersed in white adipose tissue, but can express UCP-1. The UCP-1 in beige adipocytes can also be activated by cold stimuli and/or certain molecules. Beige adipocytes can be recruited or induced to form from white adipocytes. Beige adipose tissue comprises brown-like adipocytes derived from white fat cells after a period of vigorous exercise. After exercise, skeletal muscle cells secrete a protein hormone called irisin. Irisin acts on white adipose tissue to increase the number of adipocytes that are rich in mitochondria and lipid droplets; a marked increase in the synthesis of UCP1; an increase in the rate of cellular respiration, but with the energy released as heat rather than fueling the synthesis of ATP. Lean adult humans have deposits of beige adipocytes in the neck and upper chest regions. When exposed to cold, beige adipocytes are activated. Obese people have few or no beige cells.

Fully stimulated brown or beige adipocytes have comparable amounts of UCP-1 suggesting similar thermogenic capacity. Thus, increasing the activity of brown adipocytes, beige adipocytes, or both holds promise for the treatment of metabolic disorders.

Adipocyte thermogenesis is the process of converting energy stored in the body into heat in organisms. There are at least three types of thermogenesis methods. The first type of thermogenesis is work-induced thermogenesis. This occurs when an organism uses its muscles to create heat through movement.

The second type of thermogenesis is thermo-regulatory thermogenesis. This type of thermogenesis produces heat to maintain an organism's body temperature through shivering. Shivering produces heat by converting the chemical energy stored in the form of ATP into kinetic energy and heat. The kinetic energy generated produces the characteristic muscle twitches associated with shivering.

The third type of thermogenesis is diet-induced thermogenesis. In diet-induced thermogenesis, a portion of dietary calories in excess of those required for immediate energy requirements are converted to heat rather than stored as adipose tissue. Some types of obesity may be related to a defect in this mechanism. Diet-induced thermogenesis includes non-shivering thermogenesis, which can occur in brown or beige adipocytes. In brown and beige adipocytes, UCP-1 starts an activation cascade, which leads to the production of heat. Non-shivering thermogenesis can be controlled by the sympathetic nervous system. The sympathetic nervous system can activate thermogenesis due to various stimuli, such as cold, the ingestion of food, and various other hormones and chemical stimuli.

Adipocyte thermogenesis and energy metabolism are reduced in obese individuals. Thus, activating brown or beige adipocytes to enhance energy expenditure is of great interest to combat obesity. In addition, conversion of existing white fat cells to brown or beige fat cells could also increase non-shivering thermogenesis and metabolism. Therefore, specific materials that stimulate brown cell development; materials that increase UCP-1 expression in various types of adipocytes; and materials that augment brown adipose tissue mass are of interest. The latter can also be increased through low temperature, hibernation and/or molecules directing brown adipocyte differentiation.

Activating compounds are any such compounds or mixtures of compounds that can activate adipose tissue to induce thermogenesis. Examples of activating compounds include certain derivatives of menthol. Other examples of activating compounds that can be used to activate adipose tissue include compounds that can be described by Formula I-III. The activating compounds can also be suitable salts of the compounds in Formula I-III.

The activating compound can be applied either as the sole active ingredient or in combination with other active ingredients. Some examples of other active ingredients include, but are not limited to, beta-3 adrenergic receptor agonists, such as mirabegron or solabegron.

The activating compound can also include metabolites and/or biologically accessible derivatives of the compounds from Formula I-III.

The activating compound can be applied to an affected area. The affected area can be throughout the body, wherein the activating compound can enter the body through ingestion of a pill comprising the activating compound. The affected area can be a targeted location on the body or locations on the body. The affected area can be an area that has an excess of adipose tissue. The affected area can have an excess of adipose tissue from the perspective or opinion of a person in need of such treatment. The affected area can have an excess of adipose tissue from the perspective or opinion of a medical professional. The affected area can have an excess of white adipose tissue. The affected area can have an excess of adipose tissue for cosmetic or aesthetic purposes. Whether the affected area can have an excess of adipose tissue for cosmetic or aesthetic purposes can be determined by a person in need of such treatment, a medical professional, or a third-party observer.

Adipose tissue can be selected from the group consisting of brown adipocytes, white adipocytes, beige adipocytes, brite adipocytes, subcutaneous adipose tissue, pericardial adipose tissue, marrow adipose tissue, and/or combinations thereof. Excess adipose tissue can be found beneath the skin (i.e. subcutaneous fat), around internal organs (i.e. visceral fat), in bone marrow (i.e. yellow bone marrow), intermuscular (i.e. within the Muscular system) and in breast tissue. An affected area can include excess adipose tissue found in subcutaneous adipose tissue, visceral adipose tissue, yellow bone marrow, intermuscular adipose tissue, and/or breast tissue.

Persons in need of such treatment can include a person or animal that has an affected area with an excess of adipose tissue. Persons in need of such treatment can have an affected area, multiple affected areas, or have a disease that is commonly associated with excess adipose tissue, such as type 1 diabetes, type 2 diabetes, insulin-resistance, dyslipidemia, irritable bowel syndrome, chronic pain, neuropathic pain, and/or inflammatory pain. Additionally, persons in need of such treatment can also include a person or lower animal that uses the treatment for body contouring, body shaping and/or obesity. Body contouring and body shaping can be used as a treatment for a single affected area or multiple affected areas.

While not wishing to be bound by scientific theory, the method can further comprise the step of activating a receptor. After the activating compound is applied to the affected area, the receptor can be activated by the activating compound. The receptor can be TRPM8, alpha adrenergic receptors, beta adrenergic receptors, gamma adrenergic receptor, PPARGC1A, and/or combinations thereof.

While not wishing to be bound by scientific theory, the method can further comprise the step of expressing a mitochondrial protein. After activating compound is applied to the affected area, the mitochondrial protein can be expressed. The mitochondrial protein can be UCP1, UCP2, PPARGC1A, PRDM 16, ACADM, CPT1A, FASN, 18S, GAPDH, and/or combinations thereof. The mitochondrial protein can be found within white adipocytes, beige adipocytes, and/or brown adipocytes.

While not wishing to be bound by scientific theory, the method can further comprise the step of activating adipose tissue to induce thermogenesis. After activating compound is applied to the affected area, adipose tissue can be activated to induce non-shivering thermogenesis. The adipose tissue can be activated to induce diet-induced thermogenesis.

While not wishing to be bound by scientific theory, the method can further comprise the steps of activating a receptor, expressing a mitochondrial protein, and/or activating adipose tissue to induce thermogenesis.

One or more adipocytes can be contacted with the activating compound using any effective means. A means for contacting the one or more adipocytes with an activating compound is any means that allows for the activating compound to directly access the adipose tissue and/or one or more adipocytes. Some suitable routes of contact include, but are not limited to, injection, buccal, enteral, inhalable, infused, intramuscular, intrathecal, intravenous, nasal, ophthalmic, oral, otic, rectal, subcutaneous, sublingual, topical, transdermal, vaginal and/or combinations thereof.

One or more adipocytes can be contacted with the activating compound can be contacted in any form suitable for safely and effectively delivering the activating compound to the affected area. Some forms the activating compound can take include, but are not limited to, tablet, pill, suppository, micro-needle patch, transdermal patch, suspension, solution, body wrap, and/or combinations thereof.

Disclosed herein is a device comprising a therapeutically effective amount of an activating compound and a means for contacting the activating compound with adipose tissue.

The device can comprise a means for contacting the activating compound with adipose tissue. Suitable means for contacting the activating compound with adipose tissue include any equipment needed to apply the activating compound to the affected area. For example, injection would be a suitable means for contacting an activating compound in a syringe with subcutaneous adipose tissue. Some examples of means for contacting the activating compound with adipose tissue include, but are not limited to injection, buccal, enteral, inhalable, infused, intramuscular, intrathecal, intravenous, nasal, ophthalmic, oral, otic, rectal, subcutaneous, sublingual, topical, transdermal, and/or combinations thereof. Oral administration can be accomplished with a pill, tablet, solution, suspension, slurry, and/or other common formulations for orally ingesting an active ingredient. Transdermal administration can be accomplished with a micro-needle patch, transdermal patch, fabric wrap, paper, seaweed wrap, and combinations thereof.

For administration to humans, or other mammalian subjects, especially pet animals, in need of such treatment, the total daily dose of the compounds of formula (I-III) depends, on the mode of administration. For example, oral administration may require a higher total daily dose, than an intravenous dose. The total daily dose may be administered in single or divided doses. A therapeutically effective amount of the activating compound is an amount of activating compound that can induce the intended effect. Some intended effects include, but are not limited to, promotion of thermogenesis, activation of adipose tissue, adipocyte differentiation, the conversion of white adipocytes to beige and/or brown adipocytes, reduction in size and/or quantity of adipose tissue, body contouring, and/or body shaping.

Some other intended effects include the treatment of obesity, type 1 diabetes, type 2 diabetes, insulin resistance, dyslipidemia, irritable bowel syndrome, chronic pain, neuropathic pain, and/or inflammatory pain.

A therapeutically effective amount means an amount of the activating compound or composition comprising the activating compound sufficient to induce a positive benefit, a health benefit, and/or an amount low enough to avoid serious side effects, i.e., to provide a reasonable benefit to risk ratio, within the sound judgment of a skilled artisan. A therapeutically effective amount can mean at least 0.01% of the activating compound, by weight of the composition, alternatively at least 0.1%. A therapeutically effective amount can be determined as the mass of the activating compound per kg of body weight of the individual. A therapeutically effective amount can mean at least 0.0001 mg/kg of body weight.

One or more adipocytes can be contacted with an activating compound in a treatment regimen. In a treatment regimen, the activating compound can be administered in a predetermined schedule. For example, an activating compound can be administered daily, weekly, monthly, and/or quarterly. Additionally, an activating compound can be administered in single and/or multiple doses.

Disclosed herein is an activating compound for use as a medicament. The activating compound can be chosen from any one of the compounds represented by Formulas I-III or suitable salts of the compounds represented by Formulas I-III. Disclosed herein is an activating compound for use in the treatment of obesity. Disclosed herein is an activating compound for use in the treatment of type 1 diabetes, type 2 diabetes, insulin-resistance, dyslipidemia, irritable bowel syndrome, chronic pain, neuropathic pain, and/or inflammatory pain. Use of an activating compound for the manufacture of a medicament for the treatment of obesity. Disclosed herein is the use of an activating compound for the manufacture of a medicament for the treatment of obesity, type 1 diabetes, type 2 diabetes, insulin-resistance, dyslipidemia, irritable bowel syndrome, and/or chronic pain, neuropathic pain, and/or inflammatory pain. Disclosed herein is an activating compound for use in body contouring. Disclosed herein is an activating compound for use in body shaping. Disclosed herein is an activating compound for use in the reduction of the size and/or quantity of adipose tissue; use of an activating compound for the manufacture of a medicament for the treatment of body contouring; use of an activating compound for the manufacture of a medicament for the treatment of body shaping; and use of an activating compound for the manufacture of a medicament for the treatment of the reduction of the size and/or quantity of adipose tissue.

The present invention is also directed to lotion compositions. A lotion composition of the present invention comprises at least one rheology structurant, which typically is a solid. The lotion composition can further comprise other optional ingredients, like surface energy modifiers. In certain embodiments, a lotion composition may comprise a rheology structurant, such as a microcrystalline wax, alkyl dimethicone, ethylene glycol dibehenate, ethylene glycol distearate, glycerol tribehenate, glycerol tristearate, and ethylene bisoleamide. A present lotion composition can contain a single rheology structurant or a mixture of two or more rheology structurants.

A lotion composition of the present invention may comprise at least one rheology structurant, which typically is a solid. The lotion composition can further comprise other optional ingredients, like surface energy modifiers. In one embodiment, a lotion composition consists essentially of, or consists of, a rheology structurant, such as a microcrystalline wax, alkyl dimethicone, ethylene glycol dibehenate, ethylene glycol distearate, glycerol tribehenate, glycerol tristearate, and ethylene bisoleamide. A present lotion composition can contain a single rheology structurant or a mixture of two or more rheology structurants.

In preparing a lotioned catamenial device according to the present invention, the lotion composition can be applied to the outer surface of the absorbent article, such as, for example, the outer surface of the topsheet. Any of a variety of application methods that distribute lubricious materials having a molten or liquid consistency can be used, such as, for example, as set forth in U.S. Pat. No. 5,968,025 and U.S. Pub. App. No. 2005/0208113. Suitable methods include but are not limited to spraying, printing (e.g., flexographic printing), coating (e.g., gravure coating), extrusion, dipping, or combinations of these application techniques, e.g., spraying the lotion composition on a rotating surface, such as a calender roll, that then transfers the composition to the outer surface of the sanitary napkin topsheet. Additionally, the manner of applying the lotion composition to a portion of a catamenial device can be such that the substrate or component does not become saturated with the lotion composition. The lotion composition can be applied to the catamenial device at any point during assembly. For example, the lotion composition can also be applied to the outer surface of the topsheet before it is combined with the other raw materials to form a finished catamenial device.

Dentifrice formulations can be prepared, using conventional methods, comprising the activating compounds described herein. For example, TABLE 1 displays that one or more activating compound can be incorporated into existing dentifrice formulations. One or more activating compounds can be incorporated into a flavor and/or perfume system in existing dentifrice formulations.

TABLE 1
Dentifrice formulations

| Ingredient | A | B | C |
|---|---|---|---|
| FD&C Blue #1 | 0.045% | 0.045% | 0.045% |
| Color Solution | | | |
| Sodium Fluoride | 0.243% | 0.243% | 0.243% |
| CARBOMER 956 | 0.300% | 0.300% | 0.300% |
| Sodium Saccharin | 0.300% | 0.300% | 0.300% |
| Sodium Phosphate, | 0.419% | 0.419% | 0.419% |
| Monobasic, | | | |
| Monohydrate | | | |
| Titanium Dioxide | 0.525% | 0.525% | 0.525% |
| Carboxymethycellulose | 0.800% | 0.800% | 0.800% |
| Sodium | | | |
| Peppermint Flavor | 1.000% | 1.000% | 1.000% |
| Activating | 0.01% | 0.01% | — |
| Compound 1 | | | |
| Activating | — | 0.01% | 0.01% |
| Compound 2 | | | |
| Tribasic Sodium | 1.100% | 1.100% | 1.100% |
| Phosphate | | | |
| Dodecahydrate | | | |
| Sodium Lauryl | 4.000% | 4.000% | 4.000% |
| Sulfate 28% | | | |
| Solution | | | |
| Silica, Dental | 15.000% | 15.000% | 15.000% |
| Type, NF | | | |
| (Zeodent 119) | | | |
| SORBITOL | 54.673% | 54.673% | 54.673% |
| SOLUTION LRS | | | |
| USP | | | |
| Water Purified, | QS* | QS* | QS* |
| USP, PhEur, JP, | | | |
| JSCI | | | |

Mouthwash formulations can be prepared, using conventional methods, comprising the activating compounds described herein. For example, TABLE 2 displays that one or more activating compound can be incorporated into existing mouthwash formulations. One or more activating compounds can be incorporated into a flavor and/or perfume system in existing mouthwash formulations.

TABLE 2
Mouthwash Formulations

| Ingredients | Control | Sample A | Sample B | Sample C |
|---|---|---|---|---|
| Cetylpyridinium Chloride USP | 0.074% | 0.074% | 0.074% | 0.074% |
| Activating compound 1 | 0 | 0.00005% | 0 | 0.0001% |
| Activating compound 2 | 0 | 0.00005% | 0.0001% | 0 |
| Superol Vegetable 99.7% Glycerine USP/FCC | 5% | 5% | 5% | 5% |
| Poloxamer 407 | 0.06% | 0.06% | 0.06% | 0.06% |
| Sucralose NF | 0.015% | 0.015% | 0.015% | 0.015% |
| Saccharin Sodium USP Granular, High Moist | 0.01% | 0.01% | 0.01% | 0.01% |
| Methyl Paraben | 0.02% | 0.02% | 0.02% | 0.02% |
| Propyl Paraben | 0.005% | 0.005% | 0.005% | 0.005% |
| Peppermint Flavor | 0.1% | 0.1% | 0.1% | 0.1% |
| Purified Water USP (Bottled) | QS | QS | QS | QS |

Gum formulations can be prepared, using conventional methods, comprising the activating compounds described herein. For example, TABLE 3 displays that one or more activating compound can be incorporated into existing gum formulations. One or more activating compounds can be incorporated into a flavor and/or perfume system in existing gum formulations.

TABLE 3
Gum Formulation

| | | Coolant |
|---|---|---|
| Ingredients | Control | Formulation |
| Activating Compound | 0.0 | 0.0001% |
| Spearmint Flavor Liquid | 3.992% | 3.992% |
| Spearmint spray dried flavor | 8% | 8% |
| Sucralose | 1% | 1% |
| Chewing gum base | QS to 25 grams | QS to 25 grams |
| Coating of finished gum | 1.245 g Xylitol/ | 1.245 g Xylitol/ |
| | stearate per 1 gram | stearate per 1 gram |
| | cube of gum | cube of gum |

Shave prep formulations can be prepared, using conventional methods, comprising the activating compounds described herein. For example, TABLE 4 displays that one or more activating compound can be incorporated into existing shave prep formulations. One or more activating compounds can be incorporated into a perfume system in existing shave prep formulations.

TABLE 4
Shave Prep Compositions

| | Samples | | | |
|---|---|---|---|---|
| Ingredients | 1 | 2 | 3 | 4 |
| Sorbitol 70% Solution | 0.97% | 0.97% | 0.97% | 0.97% |
| Glycerin | 0.49% | 0.49% | 0.49% | 0.49% |
| Water | QS | QS | QS | QS |
| Hydroxyethyl | 0.49% | 0.49% | 0.49% | 0.49% |
| cellulose¹ | | | | |
| PEG-90M² | 0.06% | 0.06% | 0.06% | 0.06% |
| PEG-23M³ | 0.05% | 0.05% | 0.05% | 0.05% |
| PTFE | 0.15% | 0.15% | 0.15% | 0.15% |
| Palmitic acid | 7.53% | 7.53% | 7.53% | 7.53% |
| Stearic acid | 2.53% | 2.53% | 2.53% | 2.53% |
| Glyceryl Oleate | 1.94% | 1.94% | 1.94% | 1.94% |
| Triethanolamine (99%) | 5.88% | 5.88% | 5.88% | 5.88% |
| Lubrajel Oil⁴ | 0.4% | 0.4% | 0.4% | 0.4% |
| Menthol | 0.15% | 0.15% | 0.15% | 0.15% |
| Fragrance | 0.87% | 0.87% | 0.87% | 0.87% |
| Other (e.g. Vit E, | 0.10% | 0.10% | 0.10% | 0.10% |
| Aloe, etc.) | | | | |
| Activating Compound 1 | — | 0.0001% | — | — |
| Activating Compound 2 | — | — | 0.0001% | 0.1% |
| Dye | 0.10% | 0.10% | 0.10% | 0.10% |
| Isopentane (and) | 2.85% | 2.85% | 2.85% | 2.85% |
| Isobutane | | | | |

¹Available as Natrosol 250 HHR from Hercules Inc., Wilmington, DE
²Available as Polyox WSR-301 from Amerchol Corp., Piscataway, NJ
³Available as Polyox WSR N-1 2K from Amerchol Corp., Piscataway, NJ
⁴Available as Microslip 519 from Micro Powders Inc., Tarrytown, NY
⁴Available from Guardian Laboratories, Hauppauge, NY
*QS refers to the term quantum sufficit, meaning as much as suffices, where the remainder of the formula hole is filled with this substance

Pre-shave prep formulations can be prepared, using conventional methods, comprising the activating compounds described herein. For example, TABLE 5 displays that one or more activating compound can be incorporated into existing pre-shave prep formulations. One or more activating compounds can be incorporated into a perfume system in existing pre-shave prep formulations.

TABLE 5
Pre-Shave Prep

| | Samples | | | |
|---|---|---|---|---|
| Ingredients | 1 | 2 | 3 | 3 |
| Water | QS | QS | QS | QS |
| Sepigel 305 (Polyacrylamide & C13-C14 | 0.50% | 0.50% | 0.50% | 0.50% |
| Isoparaffin & Laureth-7) | | | | |
| Polyox N13K (PEG-23M) | 0.50% | 0.50% | 0.50% | 0.50% |
| Natrosol 250 HHR (HEC) | 0.80% | 0.80% | 0.80% | 0.80% |
| Glycerin 99.7% USP/Fcc | 5.0% | 5.0% | 5.0% | 5.0% |
| Brij 35 (Laureth 23) | 2.0% | 2.0% | 2.0% | 2.0% |
| Disodium EDTA | 0.10% | 0.10% | 0.10% | 0.10% |
| Perfume | 0.15% | 0.15% | 0.15% | 0.15% |
| Glydant Plus | 0.20% | 0.20% | 0.20% | 0.20% |
| Menthol | 0.04% | 0.04% | 0.04% | 0.04% |
| Activating Compound 1 | 0.000001% | 0.000001% | 0.000001% | 0.1% |
| Activating Compound 2 | 0.000001% | 0.000001% | 0.000001% | 0.1% |

Shampoo formulations can be prepared, using conventional methods, comprising the activating compounds described herein. For example, TABLE 6 displays that one or more activating compound can be incorporated into existing shampoo formulations. One or more activating compounds can be incorporated into a perfume system in existing shampoo formulations.

TABLE 6
Shampoo Formulations

| | Samples | | | | | | | |
|---|---|---|---|---|---|---|---|---|
| Ingredients | A | B | C | D | E | F | G | H |
| Sodium Laureth Sulfate (SLE₃S) | | | | | | 6 | 6 | 6 |
| Sodium Laureth Sulfate (SLE₁S) | 10.5 | 10.5 | 12 | 12 | 12 | | | |
| Sodium Lauryl Sulfate (SLS) | 1.5 | 1.5 | | | | 7 | 7 | 7 |
| Cocamidopropyl Betaine | 1 | 1.25 | 1.5 | 1.5 | 1.5 | 1 | 1 | 1 |
| Cocamide MEA | 1 | 1.5 | 1.5 | 1.5 | 1.5 | | | |
| Glycol Distearate | 1.5 | 1.5 | 1.5 | 1.5 | 1.5 | 1.5 | 1.5 | 1.5 |
| Zinc Pyrithione | 1 | 1 | 1 | 1 | 1 | 1 | 1 | 1 |
| Zinc Carbonate | 1.61 | 1.61 | 1.61 | 1.61 | 1.61 | 1.61 | 1.61 | 1.61 |
| Menthol | 0.45 | 0.45 | 0.45 | 0.45 | 0.45 | 0.45 | 0.45 | |
| Activating Compound | 0.09 | 0.09 | 0.09 | 0.09 | 0.09 | 0.09 | 0.09 | 0.09 |
| Fragrance | 0.7 | 0.7 | 0.7 | 0.7 | 0.7 | 0.7 | 0.7 | 0.7 |
| Guar Hyrdroxypropyltrimonium Chloride (LMW) | 0.3 | 0.3 | 0.3 | 0.3 | 0.3 | 0.23 | 0.23 | 0.23 |
| Polyquaternium-10 (HMW) | | | 0.1 | 0.1 | 0.1 | | | |
| Polyquaternium 76 (AM:Triquat) | 0.01 | 0.01 | | | | | 0.01 | 0.01 |
| Stearyl Alcohol | | | | 1.29 | | | | |
| Cetyl Alcohol | | | | 0.71 | | | | |
| Dimethicone | 1.7 | 0.8 | 0.8 | 0.8 | 1.7 | 0.8 | 0.8 | 0.8 |
| Hydrochloric acid | QS | QS | QS | QS | QS | QS | QS | QS |
| Preservative | 0.05 | 0.05 | 0.05 | 0.05 | 0.05 | 0.05 | 0.05 | 0.05 |
| Sodium Chloride | QS | QS | QS | QS | QS | QS | QS | QS |
| Sodium Xylene Sulfonate | QS | QS | QS | QS | QS | QS | QS | QS |
| Sodium Benzoate (22) | 0.27 | 0.27 | 0.27 | 0.27 | 0.27 | 0.27 | 0.27 | 0.27 |
| Water and Minors (QS to 100%) (23) | QS | QS | QS | QS | QS | QS | QS | QS |

### EXAMPLES

The following non-limiting EXAMPLES represent molecules synthesized using one or more methods of the present invention. All EXAMPLES were run at room temperature (RT), standard pressure and atmosphere, unless otherwise noted. The H₂O used in the EXAMPLES was deionized H₂O, unless otherwise noted.

### Example 1. Synthesis of (S)-2-((2-benzamido-1-phenylethyl)amino)-2-oxoethan-1-aminium Chloride (1a)

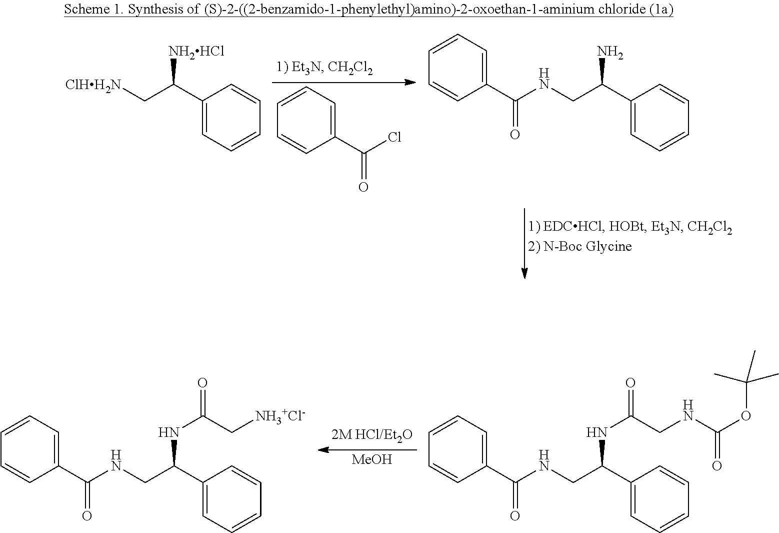

In a 300 mL 2-neck round-bottomed flask equipped with stir bar, N₂ inlet for inert gas and an addition funnel, (S)-1-phenylethane-1,2-diamine dihydrochloride (See US20170057911 for synthesis from L-phenylalanine, 2.34 gram, 11.35 mmol) was dissolved in anhydrous methylene chloride (CH₂Cl₂, 100 mL) and triethylamine (CAS#121-44-8, 6 mL, 82 mmol). The solution was cooled to 0° C. and benzoyl chloride (CAS#98-88-4, 1.58 g, 11.28 mmol) in CH₂Cl₂ (20 mL) was added dropwise wise via the addition funnel over 30 minutes. The reaction was warmed to RT and stirred 24 hr. The reaction contents were poured onto 500 mL H₂O and the layers were separated using a 1 L separatory funnel. The organic layer was washed with brine (3×300 mL) and dried over Na₂SO₄. The organic layer was concentrated under vacuum (5-10 mm Hg) and the residue was chromatographed on SiO₂ (10% MeOH/CH₂Cl₂) to provide the (S)—N-(2-amino-2-phenylethyl)benzamide (1b) as a yellow solid after concentration. 240 mg. LC/MS (241, M+H⁺).

A 250 mL 3-neck round bottom flask equipped with a condenser with an outlet to a Firestone valve (positive nitrogen pressure) and a magnetic stir bar was charged with Boc-Gly (CAS#4530-20-5, 0.160 g, 0.913 mmol), HOBt (CAS#2592-95-2, 0.140 g, 1.0375 mmol), EDC-HCl (CAS#25952-53-8, 0.198 g, 1.0375 mmol), and 100 mL anhydrous CH₂Cl₂. The solution was stirred at RT under nitrogen and triethylamine (CAS#121-44-8, 300 μL, 4.08 mmol) was added. (S)—N-(2-amino-2-phenylethyl)benzamide (0.200 g, 0.83 mmol) dissolved in 40 mL of CH₂Cl₂ was added to the heterogeneous mixture. The heterogeneous mixture was stirred at RT under a nitrogen atmosphere for 3 hr. The reaction mixture was transferred to a 1 L separatory funnel containing CH₂Cl₂ (100 mL) and H₂O (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous Na₂SO₄, filtered, and concentrated under vacuum at 38° C. to give N-Boc protected carboxamide as a white solid which was used in the next step without further purification. 185 mg.

In a 100 mL round bottom flask equipped with a stir bar, the protected carboxamide was combined with MeOH (40 mL). 2M HCl/Et₂O (CAS#7647-01-0, 10 mL) was added to the protected carboxamide solution and the reaction was stirred 24 h at RT. The solvent was removed under vacuum (5-10 mm Hg) and the residue was triturated with Et₂O (3×50 mL). The resulting solid was filtered and dried for 24 hr at RT under vacuum (5-10 mm Hg) to provide (S)-2-((2-benzamido-1-phenylethyl)amino)-2-oxoethan-1-aminium chloride (1a) as a white solid. 75 mg. LC/MS (ESI-Cl)⁺ 299. ¹H NMR (D₂O/300 MHz): δ 7.40-7.10 (m, 10H); 5.20 (m, 1H); 4.0-3.5 (m, 4H). ¹³C NMR (D₂O/100 MHz): δ 171, 166.5, 138.5, 133.2, 128.9, 126.9, 126.6, 53.5, 43.8, 40.4

Scheme 1 was modified, by changing the appropriate amino acids incorporated (either as the starting material or in the second synthesis step of Scheme 1), to obtain the following compounds: (S)—N-(2-amino-2-phenylethyl) benzamide (1b), (R)-1-(((S)-2-benzamido-1-phenylethyl)amino)-1-oxopropan-2-aminium chloride (1c), and (S)-1-(((S)-2-benzamido-1-phenylethyl)amino)-1-oxopropan-2-aminium chloride (1d). 1a, 1b, 1c, and 1d are available in TABLE 7.

### Example 2. Synthesis of (S)-2-((2-(nicotinamido)-1-phenylethyl)amino-2-oxoethan-1-aminium Chloride (2a)

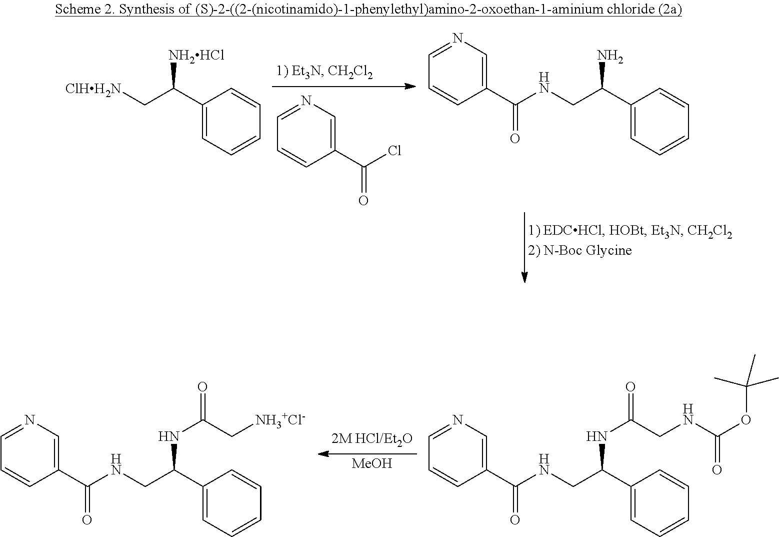

In a 300 mL 2-neck round-bottomed flask equipped with stir bar, N₂ inlet for inert gas and an addition funnel, (S)-1-phenylethane-1,2-diamine dihydrochloride (See US20170057911 for synthesis from L-phenylalanine, 3.24 gram, 15.60 mmol) was dissolved in anhydrous CH₂Cl₂ (100 mL) and triethylamine (CAS#121-44-8, 7 mL, 95 mmol). The solution was cooled to −78° C. and nicotinoyl chloride (CAS#10400-19-8, 2.0 g, 14.80 mmol) in CH₂Cl₂ (20 mL) was added dropwise wise via the addition funnel over 30 minutes. The reaction was warmed to RT and stirred 24 hr. The reaction contents were poured onto 500 mL H₂O and the layers were separated using a 1 L separatory funnel. The organic layer was washed with brine (3×300 mL) and dried over Na₂SO₄. The organic layer was concentrated under vacuum (5-10 mm Hg) and the residue was chromatographed on SiO₂ (10% MeOH/CH₂Cl₂) to provide the (S)—N-(2-amino-2-phenylethyl)nicotinamide (2b) as a white foam after concentration. 580 mg. LC/MS (243, M-Cl).

A 250 mL 3-neck round bottom flask, equipped with a condenser with an outlet to a Firestone valve (positive nitrogen pressure) and a magnetic stir bar, was charged with Boc-Gly (CAS#4530-20-5, 0.180 g, 1.02 mmol), HOBt (CAS#2592-95-2, 0.140 g, 1.0375 mmol), EDC-HCl (CAS#25952-53-8, 0.200 g, 1.05 mmol), and 100 mL anhydrous CH₂Cl₂. The solution was stirred RT under nitrogen and triethylamine (CAS#121-44-8, 500 μL, 6.80 mmol) was added. (S)—N-(2-amino-2-phenylethyl)nicotinamide (0.200 g, 0.82 mmol) dissolved in 20 mL of CH₂Cl₂ was added to the heterogeneous mixture. The heterogeneous mixture was stirred at RT under a nitrogen atmosphere for 3 hr. The reaction mixture was transferred to a 1 L separatory funnel containing CH₂Cl₂ (100 mL) and H₂O (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous Na₂SO₄, filtered, and concentrated under vacuum at 38° C. to give N-Boc protected carboxamide as a white solid which was used in the next step without further purification.

In a 100 mL round bottom flask equipped with a stir bar, the protected carboxamide was combined with MeOH (25 mL). To this solution was added 2M HCl/Et₂O (CAS#7647-01-0, 5 mL) and the reaction was stirred 24 hr at RT. The solvent was removed under vacuum (5-10 mm Hg) and the residue was triturated with Et₂O (3×50 mL). The resulting solid was filtered and dried for 24 hr at RT under vacuum (5-10 mm Hg) to provide (S)-2-((2-(nicotinamido)-1-phenylethyl)amino-2-oxoethan-1-aminium chloride (2a) as a white solid. 143 mg. LC/MS (ESI) (M−HCl) 299. ¹H NMR (D₂O/300 MHz): δ 9.50 (2s, 1H); 8.80 (m, 2H); 8.00 (m, 1H); 7.50 (m, 5H); 5.40 (m, 1H); 4.80 (m, 2H); 3.80 (m, 2H). ¹³C NMR (D₂O/100 MHz): δ 168, 162, 146, 144, 142, 138, 130, 128.5, 128.3, 128, 126, 67, 56, 43.

Scheme 2 was modified. by changing the appropriate amino acids incorporated (either as the starting material or in the second synthesis step of Scheme 2, to obtain the following compounds: (S)—N-(2-amino-2-phenylethyl)nicotinamide (2b) (¹H NMR (D₂O/300 MHz): δ 9.00 (m, 1H); 8.50 (m, 1H); 8.10 (m, 1H); 7.50 (m, 5H); 4.50 (m, 1H); 3.80 (m, 2H); 3.50 (m, 3H, NH+NH₂ exchangeable). ¹³C NMR (D₂O/100 MHz): δ 166, 152, 148, 139, 135, 130, 128, 127, 126, 124, 55, 48), (R)-1-(((S)-2-(nicotinamide)-1-phenylethyl)amino)-1-oxopropan-2-aminium chloride (2c), and (S)-1-(((S)-2-(nicotinamido)-1-phenylethyl)amino)-1-oxopropan-2-aminium chloride (2d). 2a, 2b, 2c, and 2d are available in TABLE 7.

### Example 3. Synthesis of (S)-2-((2-(nicotinoyloxy)-1-phenylethyl)amino)-2-oxoethan-1-aminium Chloride (3a)

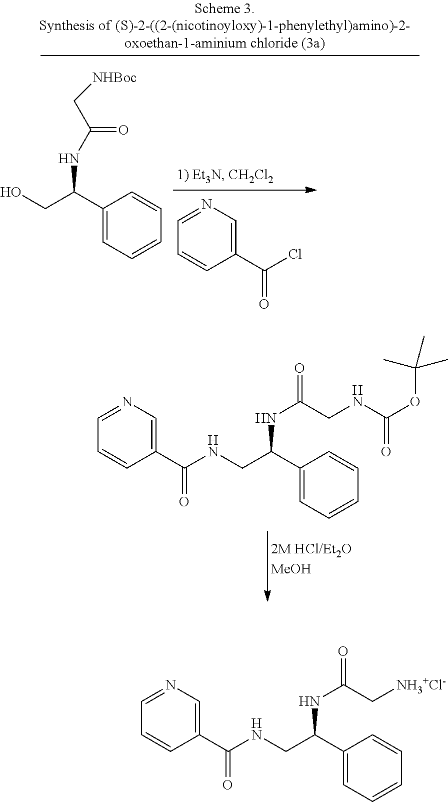

In a 300 mL 2-neck round-bottomed flask, equipped with stir bar, N₂ inlet for inert gas and an addition funnel, tert-butyl (S)-2-((2-hydroxy-1-phenylethyl)amino)-2-oxoethyl)carbamate (CAS#674789-73-2, 3.6 g, 11.68 mmol) was combined with anhydrous CH₂Cl₂ (100 mL) and triethylamine (CAS#121-44-8, 5.2 mL, 70.1 mmol). The solution was cooled to 0° C. and nicotinoyl chloride (CAS#10400-19-8, 1.5 g, 10.63 mmol) in CH₂Cl₂ (20 mL) was added dropwise wise via the addition funnel over 30 minutes. The reaction was warmed to RT and stirred 24 hr. The reaction contents were poured onto 500 mL H₂O and the layers were separated using a 1 L separatory funnel. The organic layer was washed with brine (3×300 mL) and dried over Na₂SO₄. The organic layer was concentrated under vacuum (5-10 mm Hg) and the residue was used without further purification

In a 250 mL round bottom flask, equipped with a stir bar, (S)-2-(2-((tert-butoxycarbonyl)amino)acetamido)-2-phenylethyl nicotinate was combined with MeOH (125 mL). 2M HCl/Et₂O (CAS#7647-01-0, 10 mL) was added to the MeOH solution and the reaction was stirred 24 hr at RT. The solvent was removed under vacuum (5-10 mm Hg) and the residue was triturated with Et₂O (3×50 mL). The resulting solid was filtered and dried 24 hr at RT under vacuum (5-10 mm Hg) to provide (S)-2-((2-(nicotinoyloxy)-1-phenylethyl)amino)-2-oxoethan-1-aminium chloride (3a) as a white solid. 1.70 grams. LC/MS (ESI)(M-Cl−) 301. ¹H NMR (D₂O/300 MHz): δ 8.80 (s, 1H); 8.50 (m, 2H); 7.80 (m, 1H); 7.0 (m, 5H); 5.0 (m, 1H); 4.40 (m, 2H); 3.80 (m, 1H); 1.0 (d, J=1 Hz, 3H). ¹³C NMR (D₂O/100 MHz): δ 171, 162, 148, 146, 144, 138, 136, 131, 130, 128, 126, 67, 55, 49, 17.

Scheme 3 was modified. by changing the appropriate amino acids incorporated (either as the starting material or in the second synthesis step of Scheme 3, to obtain the following compounds: (R)-1-(((S)-2-(nicotinoyloxy)-1-phenylethyl)amino)-1-oxopropan-2-aminium chloride (3b), (S)-1-(((S)-2-(nicotinoyloxy)-1-phenylethyl)amino)-1-oxopropan-2-aminium chloride (3c), and (S)-2-amino-2-phenylethyl nicotinate (3d). 3a, 3b, 3c, and 3d are available in TABLE 7.

### Example 4. Synthesis of (S,E)-2-((2-(3,7-dimethylocta-2,6-dienamido)-1-phenylethyl)amino-2-oxoethan-1-aminium Chloride (4a)

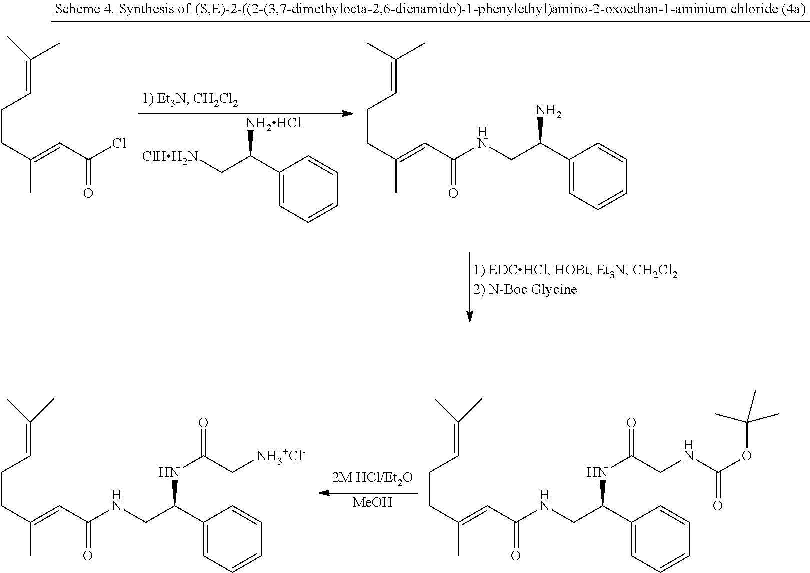

In a 500 mL 3-neck round-bottomed flask equipped with stir bar, N₂ inlet for inert gas and an addition funnel, (S)-1-phenylethane-1,2-diamine dihydrochloride (See US20170057911 for synthesis from L-phenylalanine, 5.6 gram, 27.05 mmol) in anhydrous CH₂Cl₂ (200 mL) and triethylamine (CAS#121-44-8, 12 mL, 164 mmol). The solution was cooled to 0° C. and (E)-3,7-dimethylocta-2,6, dienoyl chloride (CAS#58558-13-7, 5.0 g, 26.88 mmol) in CH₂Cl₂ (100 mL) was added dropwise wise via the addition funnel over 25 minutes. The reaction was warmed to RT and stirred 5 days. The reaction contents were poured onto 300 mL H₂O/1N HCl and the layers were separated using a 1 L separatory funnel. The organic layer was washed with 1N HCl (3×200 mL), brine (3×300 mL), and dried over Na₂SO₄. The organic layer was concentrated under vacuum (5-10 mm Hg) and the residue was chromatographed on SiO₂ (10% MeOH/CH₂Cl₂) to provide the (S,E)-N-(2-amino-2-phenylethyl)-3,7-dimethylocta-2,6-dienamide as a dark red oil after concentration (4d). 1.70 g. LC/MS (288, M+H⁺).

A 250 mL 3-neck round bottom flask, equipped with a condenser with an outlet to a Firestone valve (positive nitrogen pressure) and a magnetic stir bar, was charged with Boc-Gly (CAS#4530-20-5, 0.633 g, 3.62 mmol), HOBt (CAS#2592-95-2, 0.600 g, 4.45 mmol), EDC-HCl (CAS#25952-53-8, 0.870 g, 4.45 mmol), and 100 mL anhydrous CH₂Cl₂. The solution was stirred at RT under nitrogen and triethylamine (CAS#121-44-8, 1.25 mL, 17 mmol) was added. (S,E)-N-(2-amino-2-phenylethyl)-3,7-dimethylocta-2,6-dienamide (0.850 g, 2.97 mmol) dissolved in 50 mL of CH₂Cl₂ was added to the heterogeneous mixture. The heterogenous mixture was stirred at RT under a nitrogen atmosphere for 3 hr. The reaction mixture was transferred to a 1 L separatory funnel containing CH₂Cl₂ (100 mL) and H₂O (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous Na₂SO₄, filtered, and concentrated under vacuum at 38° C. to give N-Boc protected carboxamide as a dark oil which was used in the next step without further purification.

In a 100 mL round bottom flask, equipped with a stir bar, the protected carboxamide was combined with MeOH (50 mL). 2M HCl/Et₂O (CAS#7647-01-0, 5 mL) was added to the solution and the reaction was stirred 24 hr at RT. The solvent was removed under vacuum (5-10 mm Hg) and the residue was triturated with Et₂O (3×50 mL). The resulting solid was filtered and dried 24 hr at RT under vacuum (5-10 mm Hg) to provide (S,E)-2-((2-(3,7-dimethylocta-2,6-dienamido)-1-phenylethyl)amino-2-oxoethan-1-aminium chloride (4a) as a reddish oil. 418 mg. LC/MS (ESI) (M-Cl−) 344. ¹H NMR (D₂O/300 MHz): δ 7.0 (m, 5H); 5.40 (m, 1H); 5.10 (m, 2H); 3.80 (s, 2H); 3.50 (m, 2H); 2.10-1.85 (m, 4H); 1.80 (s, 3H); 0.60 (s, 6H).

Scheme 4 was modified. by changing the appropriate amino acids incorporated (either as the starting material or in the second synthesis step of Scheme 4, to obtain the following compounds: (R)-1-(((S)-2-((-3,7-dimethylocta-2,6-dienamido)-1-phenylethyl)amino)-1-oxopropan-2-aminium chloride (4b) (¹H NMR (D₂O/300 MHz): δ 8.40 (br s, 2H); 7.0-6.90 (m, 5H); 5.20 (m, 1H), 4.80 (m, 1H); 3.60 (m, 2H); 3.40 (m, 3H); 2.50 (s, 3H); 1.70-1.10 (m, 4H) 1.25 (d, J=3 Hz, 6H); 0.75 (s, 6H)), (S)-1-(((S)-2-((-3,7-dimethylocta-2,6-dienamido)-1-phenylethyl)amino)-1-oxopropan-2-aminium chloride (4c), and (S,E)-N-(2-amino-2-phenylethyl)-3,7-dimethylocta-2,6-dienamide (4d). 4a, 4b, 4c, and 4d are available in TABLE 7.

### Example 5. Synthesis of (R)-1-(((R)-3-(((E)-3,7-dimethylocta-2,6-dien-1-yl)oxy)-3-oxo-1-phenylpropyl)amino)-1-oxopropan-2-aminium Chloride (5a)

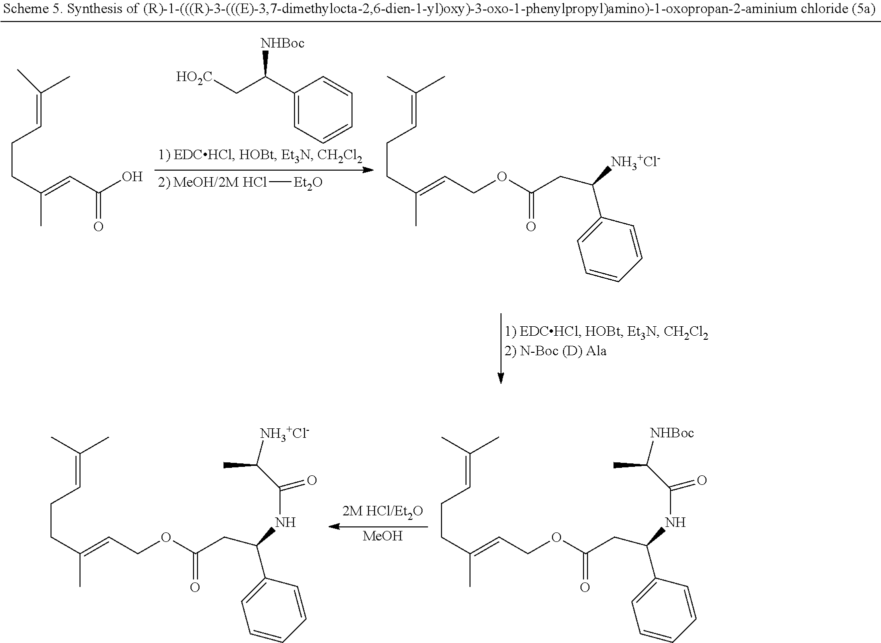

A 250 mL 3-neck round bottom flask, equipped with a condenser with an outlet to a Firestone valve (positive nitrogen pressure) and a magnetic stir bar, was charged with Boc-N-phenylpropionic acid (CAS#161024-80-2, 1.0 g, 3.77 mmol), HOBt (CAS#2592-95-2, 0.610 g, 4.51 mmol), EDC-HCl (CAS#25952-53-8, 0.880 g, 4.51 mmol), and 100 mL anhydrous CH₂Cl₂. The solution was stirred at RT under nitrogen and triethylamine (CAS#121-44-8, 2 mL, 27.22 mmol) was added. Geraniol (CAS#106-24-1, 0.600 g, 3.89 mmol) dissolved in 50 mL of CH₂Cl₂ was added to the heterogeneous mixture. The heterogenous mixture was stirred at RT under a nitrogen atmosphere for 3 hr. The reaction mixture was transferred to a 1 L separatory funnel containing CH₂Cl₂ (100 mL) and H₂O (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous Na₂SO₄, filtered, and concentrated under vacuum at 38° C. to give N-Boc protected carboxamide as a white solid which was used without further purification.

In a 100 mL round bottom flask, equipped with a stir bar, the protected carboxamide was combined with MeOH (50 mL). 2M HCl/Et₂O (CAS#7647-01-0, 5 mL) was then added and the reaction was stirred 24 hr at RT. The solvent was removed under vacuum (5-10 mm Hg) and the residue was triturated with Et₂O (3×50 mL). The resulting solid was filtered and dried 24 hr at RT under vacuum (5-10 mm Hg) to provide (R,E)-3-((3,7-dimethylocta-2,6-dien-1-yl)oxy)-3-oxo-1-phenylpropan-1-aminium chloride (5c) as a white solid. 380 mg. LC/MS (ESI) (MH+-Cl−) 302.

A 250 mL 3-neck round bottom flask, equipped with a condenser with an outlet to a Firestone valve (positive nitrogen pressure) and a magnetic stir bar, was charged with N-Boc-(D)-Ala (CAS#7764-95-6, 0.274 g, 1.45 mmol), HOBt (CAS#2592-95-2, 0.200 g, 1.49 mmol), EDC-HCl (CAS#25952-53-8, 0.282 g, 1.45 mmol), and 100 mL anhydrous CH₂Cl₂. The solution was stirred at RT under nitrogen and triethylamine (CAS#121-44-8, 0.600 mL, 4.30 mmol) was added. (R,E)-3-((3,7-dimethylocta-2,6-dien-1-yl)oxy)-3-oxo-1-phenylpropan-1-aminium chloride (0.380 g, 1.13 mmol) dissolved in 50 mL of CH₂Cl₂ was added to the heterogeneous. The heterogeneous mixture was stirred at RT under a nitrogen atmosphere for 3 hr. The reaction mixture was transferred to a 1 L separatory funnel containing CH₂Cl₂ (100 mL) and water (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous NaSO₄, filtered, and concentrated under vacuum at 38° C. to give N-Boc protected carboxamide as a white solid which was used in the next step without further purification.

In a 100 mL round bottom flask, equipped with a stir bar, the protected carboxamide was combined with MeOH (50 mL). 2M HCl/Et₂O (CAS#7647-01-0, 5 mL) was then added to the solution and the reaction was stirred 24 hr at RT. The solvent was removed under vacuum (5-10 mm Hg) and the residue was triturated with Et₂O (3×50 mL). The resulting solid was filtered and dried 24 hr at RT under vacuum (5-10 mm Hg) to provide (R)-1-(((R)-3-(((E)-3,7-dimethylocta-2,6-dien-1-yl)oxy)-3-oxo-1-phenylpropyl)amino)-1-oxopropan-2-aminium chloride (5a) as a white solid. 200 mg. LC/MS (ESI) (MH⁺-Cl−) 374.

Scheme 5 was modified by changing the appropriate amino acids incorporated (either as the starting material or in the second synthesis step of Scheme 5, to obtain the following compounds: (S)-1-(((R)-3-(((E)-3,7-dimethylocta-2,6-dien-1-yl)oxy)-3-oxo-1-phenylpropyl)amino)-1-oxopropan-2-aminium chloride (5b), (R,E)-3-((3,7-dimethylocta-2,6,dien-1-yl)oxy)-3-oxo-1-phenylpropan-1-aminium chloride (5c), and (R,E)-2-((3,7-dimethylocta-2,6-dien-1-yl)oxy)-3-oxo-1-phenylpropyl)amino)-2-oxoethan-1-aminium chloride (5d). 5a, 5b, 5c, and 5d are available in TABLE 7.

### Example 6. Synthesis of (S)-2-((2-(2-isopropyl-5-methylbenzamido)-1-phenylethyl)amino-2-oxoethan-1-aminium Chloride (6a)

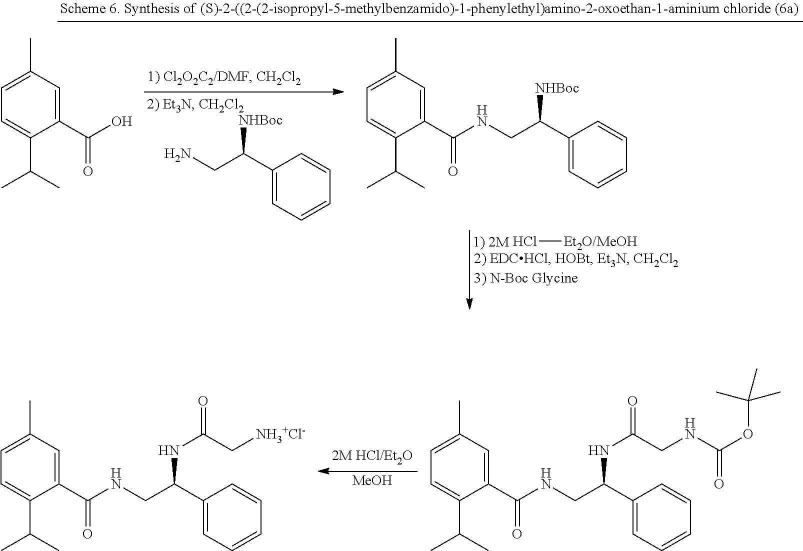

In a 250 mL round-bottomed flask, equipped with stir bar and N₂ inlet for inert gas, 2-isopropyl-5-methylbenzoic acid (CAS#4424-25-3, 2.5 g, 14.04 mmol) was dissolved in anhydrous CH₂Cl₂ (100 mL). 1 mL of DMF was added and then followed by oxalyl chloride (CAS#79-37-8, 1.5 mL, 16.85 mmol) drop wise over 20 minutes. Copious gas evolution ensued upon addition. After the addition was complete, the reaction was stirred 24 hr at RT. After 24 hr, solid tert-butyl (S)-(2-amino-1-phenylethyl) carbamate (CAS#137102-30-8, 3.5 g, 14.22 mmol) was added followed by triethylamine (CAS#121-44-8, 9.8 mL, 133 mmol). The solution reaction was stirred 24 hr. The reaction contents were poured onto 500 mL H₂O and the layers were separated using a 1 L separatory funnel. The organic layer was washed with brine (3×300 mL) and dried over Na₂SO₄. The organic layer was concentrated under vacuum (5-10 mm Hg) to provide tert-butyl (S)-(2-(2-isopropyl-5-methylbenzamido)-1-phenylethyl) carbamate as a tan solid, which was used without further purification. 2.3 g.

In a 250 mL round bottom flask, equipped with a stir bar, tert-butyl (S)-(2-(2-isopropyl-5-methylbenzamido)-1-phenylethyl) carbamate was combined with MeOH (100 mL). 2M HCl/Et₂O (CAS#7647-01-0, 5 mL) was added and the reaction was stirred 24 hr at RT. The solvent was removed under vacuum (5-10 mm Hg) and the residue was triturated with Et₂O (3×50 mL). The resulting solid was filtered and dried 24 hr at RT under vacuum (5-10 mm Hg) and used without further purification. 600 mg.

A 250 mL, 3-neck round bottom flask, equipped with a condenser with an outlet to a Firestone valve (positive nitrogen pressure) and a magnetic stir bar, was charged with Boc-Gly (CAS#4530-20-5, 0.292 g, 0.1.67 mmol), HOBt (CAS#2592-95-2, 0.220 g, 1.62 mmol), EDC-HCl (CAS#25952-53-8, 0.311 g, 1.59 mmol), and 100 mL anhydrous CH₂Cl₂. The solution was stirred at RT under nitrogen and triethylamine (CAS#121-44-8, 600 μL, 8.16 mmol) was added. (S)-2-(2-isopropyl-5-methylbenzamido)-1-phenylethan-1-aminium chloride (0.432 g, 1.3 mmol) was added to the heterogeneous solution. The mixture was stirred at RT under a nitrogen atmosphere for 24 hr. The reaction mixture was transferred to a 1 L separatory funnel containing CH₂Cl₂ (100 mL) and H₂O (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous Na₂SO₄, filtered, and concentrated under vacuum at 38° C. to give N-Boc protected carboxamide as a white solid which was used in the next step without further purification.

In a 100 mL round bottom flask, equipped with a stir bar, the protected carboxamide was combined with MeOH (50 mL). 2M HCl/Et₂O (CAS#7647-01-0, 5 mL) was added and the reaction was stirred 24 hr at RT. The solvent was removed under vacuum (5-10 mm Hg) and the residue was triturated with Et₂O (3×50 mL). The resulting solid was filtered and dried 24 hr at RT under vacuum (5-10 mm Hg) to provide (S)-2-((2-(2-isopropyl-5-methylbenzamido)-1-phenylethyl)amino-2-oxoethan-1-aminium chloride (6a) as a white solid. 298 mg. LC/MS(ESI) (M+H-Cl) 354. ¹H NMR (D₂O/300 MHz): δ 8.90 (br s, 1H); 8.30 (br s, 2H); 7.50-7.30 (m, 5H); 7.20 (m, 2H), 6.90 (m, 1H); 5.40 (m, 1H); 3.85 (m, 4H); 2.80 (m, 1H); 2.10 (s, 3H); 1.20 (d, J=3 Hz, 6H). ¹³C NMR (D₂O/100 MHz): δ 172.7, 165.5, 142.9, 139.2, 135.5, 135.0, 130.3, 128.4, 127.7, 129.9, 126.7, 126.5, 126.4, 53.5, 47.1, 40.3, 29.4, 23.2, 19.5.

Scheme 6 was modified by changing the appropriate amino acids incorporated (either as the starting material or in the second synthesis step of Scheme 6, to obtain the following compounds: (S)-2-(2-isopropyl-5-methylbenzamido)-1-phenylethan-1-aminium chloride (6b) (¹H NMR (D₂O/300 MHz): δ 7.05 (m, 5H); 6.75 (m, 2H); 6.25 (s, 1H); 4.10 (m, 1H); 3.50 (dd, J=3 Hz, 7 Hz, 2H); 2.00 (m, 1H); 1.75 (s, 3H); 0.50 (dd, J=3 Hz, 7 Hz, 6H). ¹³C NMR (D₂O/100 MHz): δ 173.8, 142.9, 135.7, 133.5, 132.2, 130, 129.7, 129.3, 127.6, 127, 126.8, 125.8, 52.8, 42.5, 29.7, 23.3, 19.7.), (R)-1-(((S)-2-(2-isopropyl-5-methylbenzamido)-1-phenylethyl)amino)-1-oxopropan-2-aminium chloride (6c) (¹H NMR (D₂O/300 MHz): δ 7.90 (br s, 1H); 7.0 (m, 5H); 6.75 (br s, 2H); 6.30 (s, 1H); 4.80 (m, 1H); 3.50 (m, 1H); 3.30 (m, 4H); 2.10 (m, 1H); 1.65 (s, 3H); 1.20 (d, J=3 Hz, 3H); 0.65 (d, J=3 Hz, 3H)), and (S)-1-(((S)-2-(2-isopropyl-5-methylbenzamido)-1-phenylethyl)amino)-1-oxopropan-2-aminium chloride (6d) (¹H NMR (D₂O/300 MHz): δ 7.90 (br s, 1H); 7.0 (m, 5H); 6.75 (br s, 2H); 6.30 (s, 1H); 4.80 (m, 1H); 3.50 (m, 1H); 3.25 (m, 4H); 2.10 (m, 1H); 1.65 (s, 3H); 1.20 (d, J=3 Hz, 3H); 0.65 (d, J=3 Hz, 3H)). 6a, 6b, 6c, and 6d are available in TABLE 7.

### Example 7. Synthesis of (R)-2-((3-(2-isopropyl-5-methylphenoxy)-3-oxo-1-phenylpropyl)amino-2-oxoethan-1-aminium Chloride (7a)

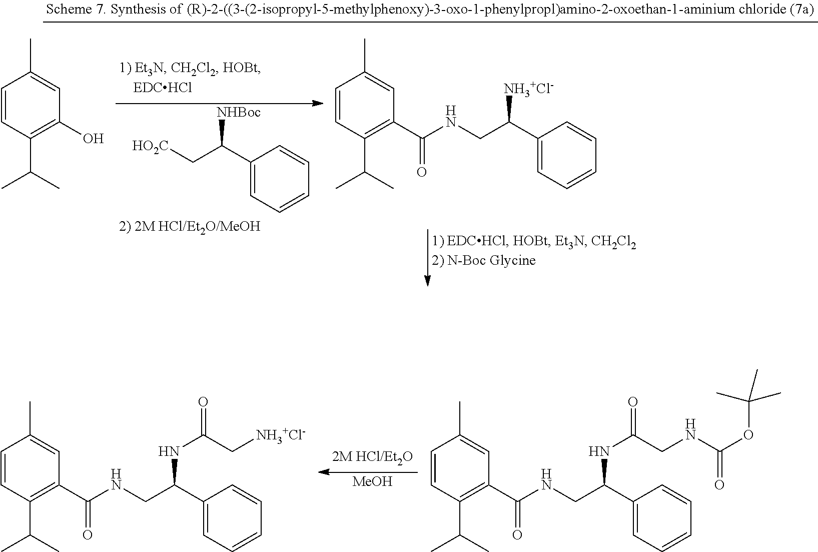

A 250 mL 3-neck round bottom flask equipped with a condenser with an outlet to a Firestone valve (positive nitrogen pressure) and a magnetic stir bar was charged with Boc-N-phenylpropionic acid (CAS#161024-80-2, 0.500 g, 1.80 mmol), HOBt (CAS#2592-95-2, 0.304 g, 2.25 mmol), EDC-HCl (CAS#25952-53-8, 0.430 g, 2.25 mmol), and 100 mL anhydrous CH₂Cl₂. The solution was stirred at RT under nitrogen and triethylamine (CAS#121-44-8, 0.75 mL, 5.40 mmol) was added. thymol (CAS#89-83-8, 0.270 g, 1.80 mmol) dissolved in 50 mL of CH₂Cl₂ was added. The heterogenous mixture was stirred at RT under a nitrogen atmosphere for 3 hr. The reaction mixture was transferred to a 1 L separatory funnel containing CH₂Cl₂ (100 mL) and H₂O (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous NaSO₄, filtered, and concentrated under vacuum at 38° C. to provide N-Boc protected ester as a white solid, which was used without further purification.

In a 250 mL round bottom flask, equipped with a stir bar, the protected carboxamide was combined with MeOH (100 mL). 2M HCl/Et₂O (CAS#7647-01-0, 5 mL) was added and the reaction was stirred 24 hr at RT. The solvent was removed under vacuum (5-10 mm Hg) and the residue was triturated with Et₂O (3×50 mL). The resulting solid was filtered, dried 24 hr under vacuum (5-10 mm Hg), and chromatographed on SiO2 (10% MeOH/CH₂Cl₂) to provide the (R)-3-(2-isopropyl-5-methylphenoxy)-3-oxo-1-phenylpropan-1-aminium chloride as a white foam. 380 mg.

A 250 mL, 3-neck round bottom flask, equipped with a condenser with an outlet to a Firestone valve (positive nitrogen pressure) and a magnetic stir bar, was charged with Boc-Gly (CAS#4530-20-5, 0.120 g, 0.685 mmol), HOBt (CAS#2592-95-2, 0.100 g, 0.740 mmol), EDC-HCl (CAS#25952-53-8, 0.130 g, 0.666 mmol), and 100 mL anhydrous CH₂Cl₂. The solution was stirred at RT under nitrogen and triethylamine (CAS#121-44-8, 200 μL, 2.16 mmol) was added. (R)-3-(2-isopropyl-5-methylphenoxy)-3-oxo-1-phenylpropan-1-aminium chloride (0.170 g, 0.51 mmol) was added to the mixture. The mixture was stirred at RT under a nitrogen atmosphere for 24 hr. The reaction mixture was transferred to a 1 L separatory funnel containing CH₂Cl₂ (100 mL) and H₂O (100 mL). The aqueous layer was separated and extracted again with 2×80 mL of CH₂Cl₂. The combined organic phases were washed with 1N HCl solution (2×50 mL), H₂O (1×50 mL), saturated NaHCO₃ solution (3×50 mL), and brine (1×50 mL). The solution was dried over anhydrous NaSO₄, filtered, and concentrated under vacuum at 38° C. to give N-Boc protected carboxamide as a white solid which was used in the next step without further purification.

In a 100 mL round bottom flask, equipped with a stir bar, the protected carboxamide was combined with MeOH (50 mL). 2M HCl/Et₂O (CAS#7647-01-0, 5 mL) was added and the reaction was stirred 24 hr at RT. The solvent was removed under vacuum (5-10 mm Hg) and the residue was triturated with Et₂O (3×50 mL). The resulting solid was filtered and dried 24 hr at RT under vacuum (5-10 mm Hg) to provide (R)-2-((3-(2-isopropyl-5-methylphenoxy)-3-oxo-1-phenylpropyl)amino-2-oxoethan-1-aminium chloride (7a) as a white solid. 75 mg. LC/MS (ESI) (M+H-Cl) 355.

Scheme 7 was modified by changing the appropriate amino acids incorporated (either as the starting material or in the second synthesis step of Scheme 7, to obtain the following compounds: (R)-1-(((R)-3-(2-isopropyl-5-methylphenoxy)-3-oxo-1-phenylpropyl)amino)-1-oxopropan-2-aminium chloride (7b), (S)-1-(((R)-3-(2-isopropyl-5-methylphenoxy)-3-oxo-1-phenylpropyl)amino)-1-oxopropan-2-aminium chloride (7c), and (R)-3-(2-isopropyl-5-methylphenoxy)-3-oxo-1-phenylpropan-1-aminium chloride (7d) (¹H NMR (D₂O/300 MHz): δ 7.80 (br s, NH2); 7.50 (m, 1H0; 7.40 (m, 5H); 7.20 (m, 1H); 6.80 (m, 1H); 6.50 (s, 1H); 4.90 (m, 1H); 3.50 (m, 2H); 2.50 (m, 1H); 2.25 (s, 3H); 1.0 (dd, J=1 Hz, 6 Hz, 6H). ¹³C NMR (D₂O/100 MHz): δ 172, 147, 143, 140, 138, 135, 132, 131, 128, 126, 124, 55, 49, 27, 23, 21). 7a, 7b, 7c, and 7d are available in TABLE 7.

### Example 8. Synthesis of (S)-2-((S)-2-aminopropanamido)-2-phenylethyl 2-isopropyl-2,3-dimethylbutanoate Hydrochloride (8a)

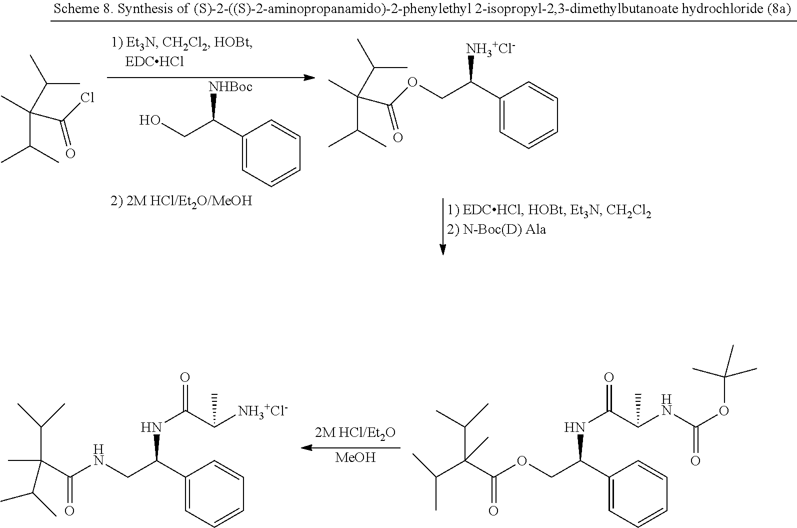

### Step 1: Synthesis of 2-isopropyl-2,3-dimethylbutanoyl Chloride

A 500 mL, 2-neck, round bottom flask immersed in an oil bath, equipped with a magnetic stir bar and fitted with a Friedrichs condenser, was charged with deionized H₂O (50 mL). Then, H₂SO₄ (80 mL) was added over 20 minutes while mixing at 300 r.p.m. The oil bath was turned on to heat and the temperature was allowed to equilibrate to 75° C. Then, N,2,3-trimethyl-2-isopropylbutamide (10.39 grams, 60.66 mmol) was added to the reaction flask.

A separate flask was charged with deionized H₂O (10 mL) and NaNO₂ (5.04 grams, 73.1 mmol). The separate solution was added to the reaction mixture containing N,2,3-trimethyl-2-isopropylbutamide over 5 minutes with observed effervescence and orange/brown fuming. The reaction was allowed to stir for 1 hr, after which time, a second addition of sodium nitrite solution (73.9 mmol of NaNO₂ in 10 mL of deionized H₂O) was added to the reaction flask. The reaction was mixed for 2 hr, after which time, a third addition of sodium nitrite solution (72.4 mmol of NaNO₂ in 10 mL of deionized H₂O) was added to the reaction. The reaction was mixed at 300 r.p.m. and at 75-80° C. for 23 hr.

The reaction was allowed to cool to RT for 1 hr following the reaction time period. The mixture was poured over approximately 600 mL of loose, crushed ice in a two liter, glass beaker, upon which there was an immediate precipitation of a white solid. The ice was allowed to melt completely and then the slurry was added to a one liter separatory funnel and extracted with 3×100 mL aliquots of Et₂O. The ether was recovered and extracted with 1×100 mL, 1×50 mL and then, 1×20 mL aliquots of 1N NaOH solution. The water layer was recovered and made acidic (pH˜2) with 200 mL of 1N HCl solution. The acidic solution was extracted with 3×100 mL aliquots of Et₂O. The organic phase was recovered and dried over anhydrous Na₂SO₄, filtered and the Et₂O removed under vacuum at 40-45° C. to give 6.2 grams of the intermediate 2-isopropyl-2,3-dimethylbutanoic acid which was used in the next synthetic step without further purification.

A 500 mL, round bottom flask equipped with a magnetic stir bar was charged with 2-isopropyl-2,3-dimethylbutanoic acid (5.07 grams, 32.04 mmol), oxalyl chloride (45.70 grams, 360 mmol), and dimethylformamide (0.023 grams, 0.31 mmol). The reaction flask was closed with a stopper and connected to a gas washing bottle containing 200 mL of 1N NaOH solution. The reaction was mixed at 250 r.p.m. and 20-25° C. under nitrogen atmosphere for 3 hr, after which time, the volatiles were removed under vacuum at 40-45° C. The residue was further evacuated to less than 5 mBar vacuum overnight to give 3.77 grams of a faint yellow liquid, 2-isopropyl-2,3-dimethylbutanoyl chloride which was used in the next synthetic step without further purification.

### Step 2: Synthesis of Tert-Butyl ((S)-1-(((S)-2-hydroxy-1-phenylethyl)amino)-1-oxopropan-2-yl)carbamate

A one liter, round bottom flask, equipped with a magnetic stir bar, was charged with (S)-(+)-2-phenylglycinol (5.10 grams, 37.2 mmol), 1H-benzo [d] [1,2,3]triazole-1-ol (4.24 grams, 31.4 mmol), N-(3-dimethylaminopropyl)-N′-ethylcarbodiimide hydrochloride (8.10 grams, 42.3 mmol), and 150 mL of tetrahydrofuran. The contents of the reaction flask were mixed at 800 r.p.m. while adding 25.25 grams (249.5 mmol) of triethylamine under nitrogen atmosphere.

A separate, one liter, round bottom flask was charged with Boc-L-Ala-OH (6.51 grams, 34.4 mmol) and 250 mL of CH₂Cl₂. This solution was transferred to a 500 mL, pressure equalizing addition funnel which was connected to the one liter reaction flask described above. The Boc-L-Ala-OH solution was added to the reaction flask over 50 minutes. The heterogeneous reaction was allowed to continue to mix at 500 r.p.m. under nitrogen atmosphere overnight.

The reaction mixture was transferred to a one liter separatory funnel following the reaction period. H₂O (200 mL) and ethyl acetate (100 mL) were added and the separatory funnel was shaken. The separated aqueous phase was transferred to an additional funnel and extracted with 2×50 mL aliquots of ethyl acetate. These extracts were subsequently recombined with the organic phase. The combined organic phases were extracted with 4×100 mL aliquots of 1N HCl solution, 3×100 mL aliquots of 1N NaOH solution and 2×100 mL aliquots of saturated NaCl solution. The organic phase was recovered and dried over anhydrous Na₂SO₄, filtered, and concentrated under vacuum at 40-45° C. to give the intermediate tert-butyl ((S)-1-(((S)-2-hydroxy-1-phenylethyl)amino)-1-oxopropan-2-yl)carbamate (Abbreviated—Boc-L-Ala-1PhE-Amd-2-ol) which was used in the next synthetic step without further purification. LC/MS (ESI) m/z=309 (MH⁺).

### Step 3: Synthesis of (S)-2-((S)-2-((tert-butoxycarbonyl)amino)propanamido)-2-phenylethyl 2-isopropyl-2,3-dimethylbutanoate

A 25 mL, round bottom flask, equipped with a magnetic stir bar, was charged with 2-isopropyl-2,3-dimethylbutanoyl chloride (0.249 grams, 1.41 mmol) prepared in Step 2. A small vial (A) was charged with triethylamine (0.165 grams, 1.63 mmol) and CH₂Cl₂ (2 mL). A small vial (B) was charged with 4-(dimethylamino) pyridine (0.166 grams, 1.36 mmol) and CH₂Cl₂ (7 mL). Small vial A and small vial B were added to the 25 mL flask. The flask was immersed in an ice bath and was allowed to equilibrate for 15 minutes while mixing at 300 r.p.m. under nitrogen atmosphere.

A separate round bottom flask was charged with tert-butyl ((S)-1-(((S)-2-hydroxy-1-phenylethyl)amino)-1-oxopropan-2-yl)carbamate (0.458 grams, 1.49 mmol) and CH₂Cl₂ (10 mL). This solution was transferred to a 10 mL, pressure equalizing addition funnel, which was connected to the 25 mL reaction flask. The Boc-L-Ala-1PhE-Amd-2-ol solution was added to the reactor over 10 minutes. The reaction was allowed to continue to mix at 250 r.p.m. in the melting ice bath and under nitrogen atmosphere overnight.

The reaction mixture was transferred to a 125 mL separatory funnel following the reaction period. The organic phase was extracted with 3×50 mL aliquots of 1N HCl solution, 2×50 mL aliquots of 1N NaOH solution and 1×50 aliquot of saturated NaCl solution. The organic phase was recovered and dried over anhydrous Na₂SO₄, filtered, and concentrated under vacuum at 35-40° C. The intermediate was further purified using flash chromatography in 75:25 v/v hexanes to ethyl acetate and collecting fractions passing through a bed of silica gel 60, 0.040-0.063 mm (230-400 mesh) approximately 155 mm long by 25 mm i.d. to give the intermediate (S)-2-((S)-2-((tert-butoxycarbonyl)amino)propanamido)-2-phenylethyl 2 isopropyl-2,3-dimethylbutanoate which was used in the next synthetic step without further purification. LC/MS (ESI) m/z=449 (MH⁺).

### Step 4: Synthesis of (S)-2-((S)-2-aminopropanamido)-2-phenylethyl 2-isopropyl-2,3-dimethylbutanoate Hydrochloride (8a)

A 25 mL, round bottom flask, equipped with a magnetic stir bar, was charged with (S)-2-((S)-2-((tert-butoxycarbonyl)amino)propanamido)-2-phenylethyl 2-isopropyl-2,3-dimethylbutanoate (0.307 grams, 0.68 mmol) and 2M HCl (10 mL) in Et₂O. The flask headspace was purged with nitrogen and the flask closed with a septum. The flask contents were mixed at 250 r.p.m. under nitrogen atmosphere for 4 hr. Following the reaction period, the volatiles were removed under vacuum and the flask contents evacuated to less than 10 mBar overnight to give 260 milligrams of the title compound, which was an off-white solid, (S)-2-((S)-2-aminopropanamido)-2-phenylethyl 2-isopropyl-2,3-dimethylbutanoate hydrochloride (8a). LC/MS (ESI) m/z=349 (MH⁺ of the free base). ¹H NMR (300 MHz, CD₃OD): δ 0.84 (d, 3H), 0.85 (d, 6H), 0.88 (d, 3H), 1.01 (s, 3H), 1.58 (d, 3H), 2.00 (m, 2H), 4.01 (q, 1H), 4.10-4.50 (m, 2H), 5.24 (dd, 1H), 7.25-7.50 (m, 5H). ¹³C NMR (75 MHz, CD₃OD): δ 13.38, 16.42, 16.44, 16.67, 17.36, 17.44, 32.30, 32.34, 48.97, 52.59, 52.86, 65.30, 126.80, 127.77, 128.42, 138.32, 169.20, 176.00.

Scheme 8 was modified by changing the appropriate amino acids incorporated (either as the starting material or in the second synthesis step of Scheme 8, to also obtain (S)-2-((R)-2-aminopropanamido)-2-phenylethyl 2-isopropyl-2,3-dimethylbutanoate hydrochloride (8b)-LC/MS (ESI) m/z=349 (MH⁺ of the free base). ¹H NMR (300 MHz, CD₃OD): δ 0.76 (d, 3H), 0.78 (d, 3H), 0.81 (d, 3H), 0.83 (d, 3H), 0.98 (s, 3H), 1.50 (d, 3H), 1.96 (m, 2H), 4.00 (q, 1H), 4.20-4.45 (m, 2H), 5.23 (t, 1H), 7.20-7.50 (m, 5H). ¹³C NMR (75 MHz, CD₃OD): δ 13.30, 16.24, 16.31, 16.39, 17.21, 17.28, 32.19, 32.30, 48.83, 52.58, 52.73, 65.01, 126.78, 127.76, 128.42, 138.51, 169.05, 175.94. 8a and 8b are available in TABLE 7.

### Example 9. Synthesis of N—((S)-2-((R)-2-aminopropanamido)-2-phenylethyl)-2-isopropyl-2,3-dimethylbutanamide (9a)

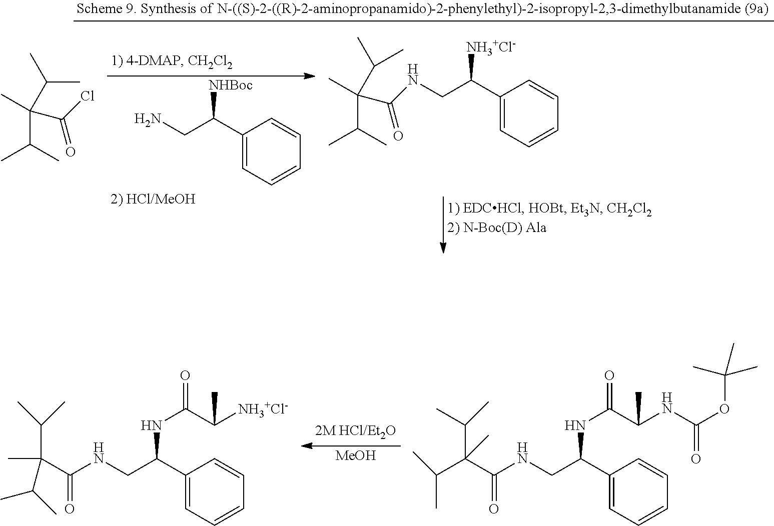

### Step 1: Synthesis of Tert-Butyl (S)-(2-(2-isopropyl-2,3-dimethylbutanamido)-1-phenylethyl)carbamate

A 25 mL round bottom flask equipped with a magnetic stir bar was charged with (S)-tert-butyl (2-amino-1-phenylethyl) carbamate (1.398 grams, 5.92 mmol), 4-(dimethylamino)pyridine (0.710 grams, 5.81 mmol), and CH₂Cl₂ (10 mL). The flask was immersed in an ice bath and was allowed to equilibrate for 15 minutes while mixing at 250 r.p.m. under nitrogen atmosphere.

A separate round bottom flask was charged with 2-isopropyl-2,3-dimethylbutanoyl chloride (0.654 grams, 3.70 mmol) in CH₂Cl₂ (5 mL). This solution was transferred to a 5 mL, pressure equalizing addition funnel which was connected to the 25 mL reaction flask. The 2-isopropyl-2,3-dimethylbutanoyl chloride solution was added to the reaction flask over 16 minutes. The heterogeneous reaction was mixed at 250 r.p.m. under nitrogen atmosphere for 3 hr.

The reaction mixture was transferred to a 250 mL separatory funnel following the reaction time period and extracted with 4×50 mL aliquots of 1N HCl, 2×50 mL aliquots of 1N NaOH and 1×50 mL aliquot of saturated NaCl solution. The organic phase was recovered and dried over anhydrous Na₂SO₄, filtered, and concentrated under vacuum at 30-35° C. to give a mixture of products which included the intermediate tert-butyl (S)-(2-(2-isopropyl-2,3-dimethylbutanamido)-1-phenylethyl)carbamate which was used in the next synthetic step without further purification. LC/MS (ESI) m/z=449 (MH⁺).

### Step 2: (S)—N-(2-amino-2-phenylethyl)-2-isopropyl-2,3-dimethylbutanamide Hydrochloride (9b)

A 100 mL round bottom flask equipped with a magnetic stir bar was charged with tert-butyl (S)-(2-(2-isopropyl-2,3-dimethylbutanamido)-1-phenylethyl)carbamate (0.601 grams, 1.60 mmol), CH₂Cl₂ (3 mL), and 50 ml of 2M HCl in diethyl ether. The flask headspace was purged with nitrogen and the flask closed with a septum. The reaction flask contents were mixed at 200 r.p.m., 20-25° C. and under nitrogen atmosphere for 20 hr. Following the reaction time period, the volatiles were removed under vacuum at 35-40° C. to give 0.299 grams of a mixture of products which included the intermediate (S)—N-(2-amino-2-phenylethyl)-2-isopropyl-2,3-dimethylbutanamide hydrochloride. This resulting mixture was purified by dissolving the residue in MeOH (15 mL). This solution was then transferred to a 250 mL separatory funnel and extracted with 1×75 mL aliquot of n-pentane. The MeOH layer was recovered and the solvent removed under vacuum at 35-40° C. to give 0.122 grams of the intermediate (S)—N-(2-amino-2-phenylethyl)-2-isopropyl-2,3-dimethylbutanamide hydrochloride (9b) which was used in the next synthetic step without further purification. ¹H NMR (300 MHz, CD₃OD): δ 0.50-1.10 (m, 15H), 1.80-2.10 (m, 2H), 3.76 (br, m, 2H), 4.52 (br, m, 1H), 7.20-7.70 (m, 5H). ¹³C NMR (75 MHz, CD₃OD): δ 12.82, 16.36, 16.39, 17.08, 17.24, 32.13, 32.19, 41.94, 51.46, 54.20, 127.78, 128.93, 129.28, 134.64, 177.86.

### Step 3: Tert-Butyl ((R)-1-(((S)-2-(2-isopropyl-2,3-dimethylbutanamido)-1-phenylethyl)amino)-1-oxopropan-2-yl)carbamate

A 25 mL round bottom flask, equipped with a magnetic stir bar, was charged with (S)—N-(2-amino-2-phenylethyl)-2-isopropyl-2,3-dimethylbutanamide hydrochloride (0.122 grams, 0.39 mmol) and CH₂Cl₂ (5 mL).

A second flask was charged with N-(3-dimethylaminopropyl)-N′-ethylcarbodiimide hydrochloride (0.152 grams, 0.79 mmol), Boc-D-Ala-OH (0.107 grams, 0.57 mmol), triethylamine (0.418 grams (4.13 mmol), and CH₂Cl₂ (10 mL). This solution was transferred to a 10 mL, pressure equalizing addition funnel which was connected to the 25 mL reaction flask. The Boc-D-Ala-OH solution was added to the solution in the 25 mL reaction flask over 6 minutes. The heterogeneous reaction was mixed at 250 r.p.m. under nitrogen atmosphere for 24 hr.

The reaction mixture was transferred to a 250 mL separatory funnel following the reaction time period and extracted with 3×50 mL aliquots of 1N HCl, 2×50 mL aliquots of 1N NaOH and 1×50 mL aliquot of saturated NaCl solution. The organic phase was recovered and dried over anhydrous Na₂SO₄, filtered, and concentrated under vacuum at 35-40° C. to give 0.048 grams of minor by-products and the intermediate tert-butyl ((R)-1-(((S)-2-(2-isopropyl-2,3-dimethylbutanamido)-1-phenylethyl)amino)-1-oxopropan-2-yl)carbamate which was used in the next synthetic step without further purification. (LC/MS (ESI) m/z=(MH⁺ 441).

### Step 4: N—((S)-2-((R)-2-aminopropanamido)-2-phenylethyl)-2-isopropyl-2,3-dimethylbutanamide (9a)

A 100 mL, round bottom flask, equipped with a magnetic stir bar, was charged with tert-butyl ((R)-1-(((S)-2-(2-isopropyl-2,3-dimethylbutanamido)-1-phenylethyl)amino)-1-oxopropan-2-yl)carbamate (0.048 grams, 0.10 mmol) and 2M HCl in Et₂O (45 mL). The flask headspace was purged with nitrogen and the flask closed with a septum. The flask contents were mixed at 200 r.p.m. under nitrogen atmosphere for 19 hr. Following the reaction time period, the volatiles were removed under vacuum at 35-40° C. to give 0.029 grams of residue that contained by-product(s) and the intermediate N—((S)-2-((R)-2-aminopropanamido)-2-phenylethyl)-2-isopropyl-2,3-dimethylbutanamide hydrochloride. This residue was further purified by adding 25 mL of 1N NaOH and extracting the solution with 3×20 mL aliquots of Et₂O. The ether layer was recovered and the solvent removed under vacuum at 35-40° C. The free amine was isolated by preparatory thin layer chromatography using 95:5 CHCl₃ to MeOH v/v with 0.15% w/v ammonium hydroxide as the eluting solvent. This technique yielded approximately 7 milligrams of the title compound N—((S)-2-((R)-2-aminopropanamido)-2-phenylethyl)-2-isopropyl-2,3-dimethylbutanamide (9a). LC/MS (ESI) m/z=348 (MH⁺). ¹H NMR (300 MHz, CD₃OD): δ 0.82 (d, 3H), 0.83 (d, 3H), 0.86 (d, 3H), 0.88 (d, 3H), 0.99 (s, 3H), 1.34 (d, 3H), 1.90-2.10 (m, 2H), 3.42-3.76 (m, 3H), 5.05 (dd, 1H), 7.20-7.50 (m, 5H). ¹³C NMR (75 MHz, CD₃OD): δ 12.87, 16.39, 16.50, 17.25, 18.91, 32.16, 32.28, 43.88, 49.78, 51.45, 53.94, 126.47, 127.29, 128.26, 139.96, 174.38, 177.55.

### Example 10. (S)-2-(2-isopropyl-2,3-dimethylbutanamido)-1-phenylethyl Glycinate Hydrochloride (10a)

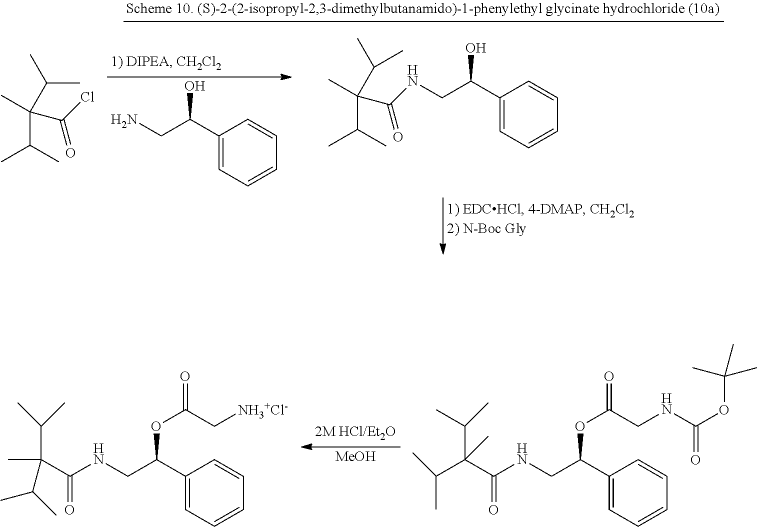

### Step 1: (S)—N-(2-hydroxy-2-phenylethyl)-2-isopropyl-2,3-dimethylbutanamide

A 25 mL round bottom flask equipped with a magnetic stir bar, was charged with (S)-(+)-2-amino-1-phenylethanol (0.580 grams, 4.23 mmol), N,N-diisopropylethyl amine (1.30 grams, 10.06 mmol), and CH₂Cl₂ (2 mL). The reaction flask containing the heterogeneous mixture was placed in an ice bath and allowed to equilibrate for 10 minutes while mixing at 300 r.p.m under nitrogen atmosphere.

A separate flask was charged with 2-isopropyl-2,3-dimethylbutanoyl chloride (0.711 grams, 4.02 mmol) and CH₂Cl₂ (5 mL). This solution was transferred to a 10 mL, pressure equalizing addition funnel which was connected to the 25 mL reaction flask. The 2-isopropyl-2,3-dimethylbutanoyl chloride solution was added to the reaction flask over 15 minutes during which time solution was achieved. The reaction was mixed at 300 r.p.m. in the melting ice bath and under nitrogen atmosphere for 4 hr.

The reaction mixture and 75 mL of Et₂O was added to a 250 mL separatory funnel following the reaction time period. The reaction mixture was extracted with 3×50 mL aliquots of 1N HCl solution, 2×50 mL aliquots of 1N NaOH solution and 2×50 mL aliquots of saturated NaCl solution. The organic phase recovered and dried over anhydrous Na₂SO₄, filtered, and concentrated under vacuum at 35-40° C. The intermediate was further purified using flash chromatography in 50:50 v/v hexanes to ethyl acetate and collecting fractions passing through a bed of silica gel 60, 0.040-0.063 mm (230-400 mesh) approximately 155 mm long by 25 mm i.d. to give 0.583 grams of the intermediate (S)—N-(2-hydroxy-2-phenylethyl)-2-isopropyl-2,3-dimethylbutanamide which was used in the next synthetic step without further purification. LC/MS (ESI) m/z=278 (MH⁺).

### Step 2: (S)-2-(2-isopropyl-2,3-dimethylbutanamido)-1-phenylethyl (Tert-Butoxycarbonyl)Glycinate

A 25 mL round bottom flask, equipped with a magnetic stir bar, was charged with (S)—N-(2-hydroxy-2-phenylethyl)-2-isopropyl-2,3-dimethylbutanamide (0.537 grams, 1.94 mmol), N-(3-dimethylaminopropyl)-N′-ethylcarbodiimide hydrochloride (0.733 grams, 3.82 mmol), and CH₂Cl₂ (4 mL). The contents of the reaction flask were mixed at 500 r.p.m. under nitrogen atmosphere.

A separate flask was charged with Boc-Gly-OH (0.561 grams, 3.20 mmol), 4-(dimethylamino)pyridine (1.278 grams, 10.46 mmol), and CH₂Cl₂ (5 mL). This solution was added to the reaction flask over 10 seconds via pipette. The reaction was mixed at 500 r.p.m. under nitrogen atmosphere overnight.

The reaction mixture and 50 mL of Et₂O was transferred to a 250 mL separatory funnel following the reaction time period. The reaction mixture was extracted with 3×20 mL aliquots of 1N HCl solution, 3×20 mL aliquots of 1N NaOH solution and 2×20 mL aliquots of saturated NaCl solution. The organic phase was recovered and dried over anhydrous Na₂SO₄, filtered, and concentrated under vacuum at 35-40° C. to give the 0.768 grams of the intermediate (S)-2-(2-isopropyl-2,3-dimethylbutanamido)-1-phenylethyl (tert-butoxycarbonyl)glycinate which was used in the next synthetic step without further purification. LC/MS (ESI) m/z=435 (MH⁺).

### Step 3: (S)-2-(2-isopropyl-2,3-dimethylbutanamido)-1-phenylethyl Glycinate Hydrochloride (10a)

A 250 mL round bottom flask equipped with a magnetic stir bar, was charged with (S)-2-(2-isopropyl-2,3-dimethylbutanamido)-1-phenylethyl (tert-butoxycarbonyl)glycinate (0.768 grams, 1.77 mmol) and 2M HCl in Et₂O (90 mL). The flask headspace was purged with nitrogen and the flask closed with a septum. The flask contents were mixed at 500 r.p.m. under nitrogen atmosphere for 20 hr. Following the reaction time period, the volatiles were removed under vacuum to give 0.784 grams of residue that contained by-product(s) and the title compound, (S)-2-(2-isopropyl-2,3-dimethylbutanamido)-1-phenylethyl glycinate hydrochloride. The product was further purified using flash chromatography. The first purification was made using 90:10 v/v CH₂Cl₂ to MeOH and collecting fractions passing through a bed of silica gel 60, 0.040-0.063 mm (230-400 mesh) approximately 155 mm long by 25 mm i.d. and gave 424 milligrams of an impure residue that contained by-product(s) and the title compound. The second purification was made using 70:30 v/v ethyl acetate to MeOH and collecting fractions passing through a similar bed of silica gel and gave 119 milligrams of an impure residue that contained by-product(s) and the title compound. The impure residue was dissolved in 25 mL of Et₂O and treated with 25 mL of 1.25 M HCl in MeOH. The solution was briefly mixed and the volatiles removed under vacuum. The remaining residue was treated with 25 mL of Et₂O which following precipitation, the Et₂O was decanted. The film like residue that remained was treated with 25 additional mL of Et₂O which was also decanted. The residual solvent was removed under vacuum overnight to give 0.104 grams of the title compound which was an off-white solid, (S)-2-(2-isopropyl-2,3-dimethylbutanamido)-1-phenylethyl glycinate hydrochloride (10a). LC/MS (ESI) m/z=335 (MH⁺ of the free base). ¹H NMR (300 MHz, CD₃OD): δ 0.83 (d, 3H), 0.85 (d, 6H), 0.87 (d, 3H), 0.99 (s, 3H), 1.90-2.10 (m, 2H), 3.40-3.80 (m, 2H), 3.90 (s, 2H), 6.00 (dd, 1H), 7.25-7.50 (m, 5H). ¹³C NMR (75 MHz, CD₃OD): δ 14.45, 15.60, 18.00, 18.02, 18.80, 33.76, 41.35, 44.92, 53.00, 53.57, 78.13, 128.09, 129.84, 129.90, 138.77, 168.03, 178.9.

### Example 11. (S)-2-((3S,5S,7S)-adamantane-1-carboxamido)-1-phenylethyl Glycinate Hydrochloride (11a)

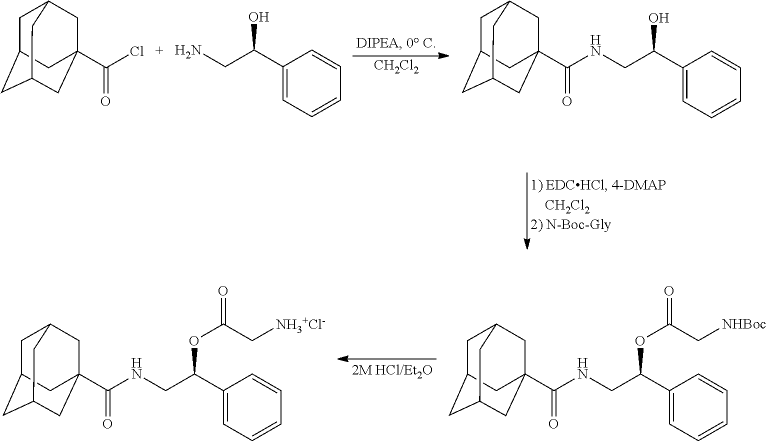

### Example 11. Synthesis of (S)-2-((3S,5S,7S)-adamantane-1-carboxamido)-1-phenylethyl Glycinate Hydrochloride (11a)

### Step 1: (3S,5S,7S)—N—((S)-2-hydroxy-2-phenylethyl) adamantane-1-carboxamide

A 25 mL round bottom flask equipped with a magnetic stir bar, was charged with (S)-(+)-2-amino-1-phenylethanol (0.485 grams, 3.53 mmol), N,N-diisopropylethyl amine (0.714 grams, 5.52 mmol), and CH₂Cl₂ (5 mL). The reaction flask containing the mixture was placed in an ice bath and allowed to equilibrate for 10 minutes while mixing at 300 r.p.m under nitrogen atmosphere.

A separate flask was charged with of 1-adamantanecarbonyl chloride (0.756 grams, 3.81 mmol) and CH₂Cl₂ (5 mL). The 1-adamantanecarbonyl chloride solution was transferred to a 10 mL, pressure equalizing addition funnel which was connected to the 25 mL reaction flask and this solution was added to the reaction flask over 12 minutes. The reaction was mixed at 300 r.p.m. in the melting ice bath and under nitrogen atmosphere for 3 hr.

The reaction mixture was added to a 125 mL separatory funnel following the reaction time period. The reaction mixture was extracted with 3×20 mL aliquots of 1N HCl solution and 1×25 mL aliquot of saturated NaCl solution. The organic phase was recovered and dried over anhydrous Na₂SO₄, filtered, and concentrated under vacuum at 35-40° C. to give 0.941 grams of the intermediate (3S,5S,7S)—N—((S)-2-hydroxy-2-phenylethyl) adamantane-1-carboxamide which was used in the next synthetic step without further purification. LC/MS (ESI) m/z=300 (MH⁺). ¹H NMR (300 MHz, CD₃OD): δ 1.65-2.10 (m, 16H), 3.28-3.51 (m, 2H), 4.76 (dd, 1H), 7.20-7.42 (m, 5H). ¹³C NMR (75 MHz, CD₃OD): δ 28.22, 36.19, 38.71, 40.40, 46.58, 72.19, 125.86, 127.21, 127.88, 142.51, 179.70

### Step 2: (S)-2-((3S,5S,7S)-adamantane-1-carboxamido)-1-phenylethyl (Tert-Butoxycarbonyl)Glycinate

A 25 mL round bottom flask equipped with a magnetic stir bar was charged with N-(3-dimethylaminopropyl)-N′-ethylcarbodiimide hydrochloride (0.624 grams, 3.26 mmol), Boc-Gly-OH, (0.453 grams, 2.59 mmol), 4-(dimethylamino)pyridine (0.974 grams, 7.97 mmol), and CH₂Cl₂ (6 mL). The contents of the reaction flask were mixed at 300 r.p.m. under nitrogen atmosphere.

A separate flask was charged with (3S,5S,7S)—N—((S)-2-hydroxy-2-phenylethyl) adamantane-1-carboxamide (0.500 grams, 1.67 mmol) and CH₂Cl₂ (5 mL). This solution was transferred to a 10 mL pressure equalizing addition funnel and added to the reaction flask over 5 minutes. The reaction was mixed at 250 r.p.m. and 20-25° C. under nitrogen atmosphere for 23 hr.

The reaction mixture was transferred to a 250 mL separatory funnel following the reaction time period. The reaction mixture was extracted with 1×100 mL aliquot of 1N HCl solution, 2×50 mL aliquots of 1N HCl, 1×50 mL aliquot of 0.1N NaOH solution and 2×50 mL aliquots of saturated NaCl solution. The organic phase was recovered and dried over anhydrous Na₂SO₄, filtered, and concentrated under vacuum at 30-35° C. to give the 0.506 grams of residue that contained by-product(s) and the intermediate (S)-2-((3S,5S,7S)-adamantane-1-carboxamido)-1-phenylethyl (tert-butoxycarbonyl)glycinate. The product was purified using flash chromatography using 60:40 v/v hexanes to ethyl acetate and collecting fractions passing through a bed of silica gel 60, 0.040-0.063 mm (230-400 mesh) approximately 155 mm long by 25 mm i.d. and gave 216 milligrams of the intermediate (S)-2-((3S,5S,7S)-adamantane-1-carboxamido)-1-phenylethyl (tert-butoxycarbonyl)glycinate which was used in the next synthetic step without further purification. LC/MS (ESI) m/z=457 (MH⁺). ¹H NMR (300 MHz, CD₃OD): δ 1.65-2.10 (m, 16H), 3.52-3.72 (m, 2H), 3.83-4.03 (m, 2H), 6.05 (dd, 1H), 7.25-7.55 (m, 5H). ¹³C NMR (75 MHz, CD₃OD): δ 28.21, 36.16, 38.73, 39.80, 40.44, 43.63, 76.35, 126.46, 128.29, 128.36, 137.13, 166.52, 179.91

### Step 3: (S)-2-((3S,5S,7S)-adamantane-1-carboxamido)-1-phenylethyl Glycinate Hydrochloride (11a)

A 25 mL round bottom flask containing a magnetic stir bar was charged with of (S)-2-((3S,5S,7S)-adamantine-1-carboxamido)-1-phenylethyl (tert-butoxycarbonyl)glycinate (0.172 grams, 0.38 mmol), CH₂Cl₂ (1 mL), and 2M HCl in Et₂O (5 mL). The flask headspace was purged with nitrogen and the flask closed with a septum. The flask contents were mixed at 300 r.p.m. under nitrogen atmosphere for 3 hours. Following the reaction time period, the volatiles were removed under vacuum to give 0.080 grams of the title compound which was a white crystalline solid, (S)-2-((3S,5S,7S)-adamantine-1-carboxamido)-1-phenylethyl glycinate hydrochloride (11a). LC/MS (ESI) m/z=358 (MH⁺ of the free base).

### Example 12. TRPM8 Protocol-FLIPR Assay

To determine whether TRPM8 is activated, the intracellular calcium ion (Ca²⁺) level was measured from transfected cells with the TRPM8 receptor sequence (SEQ ID NO: 1). HEK-293 (human embryonic kidney) cells stably transfected with human TRPM8 were grown in 15 mL growth medium (high glucose DMEM (Dulbecco's Modification of Eagle's Medium) supplemented with 10% FBS (fetal bovine serum), 100 μg/mL penicillin/streptomycin, 5 μg/mL blasticindin, and 100 μg/mL zeocin) in a 75 cm² flask for 3 days at 37° C. in a mammalian cell culture incubator (Forma Scientific Model 3110, Marietta, Ohio) set at 5% CO₂. Cells were detached with addition of 2 mL of trypsin-EDTA buffer (GIBCO® 25200, Invitrogen, Grand Island, N.Y.) for about 2-3 min. Trypsin was inactivated by addition of 8 mL growth medium. Cells were transferred to a 50 mL tube and centrifuged at 850 rpm for 3 minutes to remove medium. After centrifugation, a pellet of cells was formed in the bottom of the tube separating them from the supernatant solution. The supernatant was discarded and the cell pellet was suspended in 1 mL of fresh growth medium to which 5 μL (12.5 μg) of Fluo-4 AM (Molecular Probes, Inc., Eugene, Oreg.) calcium indicator was added and incubated for 30 min with gentle shaking. Fluo-4 AM is a fluorescent dye used for quantifying cellular Ca²⁺ concentrations in the 100 nM to 1 μM range. At the end of 30 minutes, 45 mL of assay buffer (1×HBSS (Hank's Balanced Salt Solution), 20 mM HEPES (4-(2-Hydroxyethyl)-1-piperazineethanesulfonic acid)) was added to wash cells and the resulting mixture was then centrifuged at 850 rpm for 3 minutes at 20° C. to remove excess buffer and Fluo-4 AM calcium indicator.

The pelleted cells were re-suspended in 10 mL assay buffer and 90 μL aliquots (50,000 cells) per well delivered to a 96-well assay plate containing 10 μL of test compounds (1 mM in assay buffer, final concentration 100 μM) or buffer control and incubated at room temperature for 30 minutes. After 30 minutes, a plate (Falcon 353219, Corning Corning N.Y.) was placed into a fluorometric imaging plate reader (FLIPR384 from Molecular Devices, Sunnyvale, Calif.) and basal fluorescence recorded (excitation wave length 488 nm and emission wave length 510 nm). Then 20 μL of 100 mM of TRPM8 agonist WS5 coolant in the assay buffer was added and fluorescence recorded. For determining the direct effect of test compounds on TRPM8, fluorescence was measured immediately after addition of each compound. Additional discussion of the FLIPR method can be found in Smart et al., Characterization using FLIPR of human vanilloid VR1 receptor pharmacology, European Journal of Pharmacology 417, 51-58 (2001) and Liu et al., Development and validation of a platelet calcium flux assay using a fluorescent imaging plate reader, Analytical Biochemistry 357, 216-224 (2006).

The magnitude of the fluorescence of the active-treated cells was compared to the magnitude of the fluorescence from a benchmark agonist (WS-5), as described above. The percentage of fluorescence as a function of active dose was plotted and a sigmoidal curve was generated. Curve fitting from this dose-response curve yielded the value for TRPM8 EC₅₀ in μM found in TABLE 8.

### Data

TABLE 7
Synthesized Compounds

| Compound Name | Compound |
|---|---|
| (S)-2-((2-benzamido-1-phenylethyl)amino)-2-oxoethan-1-aminium chloride | (1a) |
| (S)-N-(2-amino-2-phenylethyl) benzamide | (1b) |
| (R)-1-(((S)-2-benzamido-1-phenylethyl)amino)-1-oxopropan-2-aminium chloride | (1c) |
| (S)-1-(((S)-2-benzamido-1-phenylethyl)amino)-1-oxopropan-2-aminium chloride | (1d) |
| (S)-2-((2-(nicotinamido)-1-phenylethyl)amino-2-oxoethan-1-aminium chloride | (2a) |
| (S)-N-(2-amino-2-phenylethyl)nicotinamide | (2b) |
| (R)-1-(((S)-2-(nicotinamido)-1-phenylethyl)amino)-1-oxopropan-2-aminium chloride | (2c) |
| (S)-1-(((S)-2-(nicotinamido)-1-phenylethyl)amino)-1-oxopropan-2-aminium chloride | (2d) |
| (S)-2-((2-(nicotinoyloxy)-1-phenylethyl)amino)-2-oxoethan-1-aminium chloride | (3a) |
| (R)-1-(((S)-2-(nicotinoyloxy)-1-phenylethyl)amino)-1-oxopropan-2-aminium chloride | (3b) |
| (S)-1-(((S)-2-(nicotinoyloxy)-1-phenylethyl)amino)-1-oxopropan-2-aminium chloride | (3c) |
| (S)-2-amino-2-phenylethyl nicotinate | (3d) |
| (S,E)-2-((2-(3,7-dimethylocta-2,6-dienamido)-1-phenylethyl)amino-2-oxoethan-1-aminium chloride | (4a) |
| (R)-1-(((S)-2-((E)-3,7-dimethylocta-2,6-dienamido)-1-phenylethyl)amino)-1-oxopropan-2-aminium | (4b) |
| chloride | |
| (S)-1-(((S)-2-((E)-3,7-dimethylocta-2,6-dienamido)-1-phenylethyl)amino)-1-oxopropan-2-aminium | (4c) |
| chloride | |
| (S,E)-N-(2-amino-2-phenylethyl)-3,7-dimethylocta-2,6-dienamide | (4d) |
| (R)-1-(((R)-3-(((E)-3,7-dimethylocta-2,6-dien-1-yl)oxy)-3-oxo-1-phenylpropyl)amino)-1-oxopropan- | (5a) |
| 2-aminium chloride | |
| (S)-1-(((R)-3-(((E)-3,7-dimethylocta-2,6-dien-1-yl)oxy)-3-oxo-1-phenylpropyl)amino)-1-oxopropan- | (5b) |
| 2-aminium chloride | |
| (R,E)-3-((3,7-dimethylocta-2,6,dien-1-yl)oxy)-3-oxo-1-phenylpropan-1-aminium chloride | (5c) |
| (R,E)-2-((3,7-dimethylocta-2,6-dien-1-yl)oxy)-3-oxo-1-phenylpropyl)amino)-2-oxoethan-1-aminium | (5d) |
| chloride | |
| (S)-2-((2-(2-isopropyl-5-methylbenzamido)-1-phenylethyl)amino-2-oxoethan-1-aminium chloride | (6a) |
| (S)-2-(2-isopropyl-5-methylbenzamido)-1-phenylethan-1-aminium chloride | (6b) |
| (R)-1-(((S)-2-(2-isopropyl-5-methylbenzamido)-1-phenylethyl)amino)-1-oxopropan-2-aminium | (6c) |
| chloride | |
| (S)-1-(((S)-2-(2-isopropyl-5-methylbenzamido)-1-phenylethyl)amino)-1-oxopropan-2-aminium | (6d) |
| chloride | |
| (R)-2-((3-(2-isopropyl-5-methylphenoxy)-3-oxo-1-phenylpropyl)amino-2-oxoethan-1-aminium | (7a) |
| chloride | |
| (R)-1-(((R)-3-(2-isopropyl-5-methylphenoxy)-3-oxo-1-phenylpropyl)amino)-1-oxopropan-2-aminium | (7b) |
| chloride | |
| (S)-1-(((R)-3-(2-isopropyl-5-methylphenoxy)-3-oxo-1-phenylpropyl)amino)-1-oxopropan-2-aminium | (7c) |
| chloride | |
| (R)-3-(2-isopropyl-5-methylphenoxy)-3-oxo-1-phenylpropan-1-aminium chloride | (7d) |
| (S)-2-((S)-2-aminopropanamido)-2-phenylethyl 2-isopropyl-2,3-dimethylbutanoate hydrochloride | (8a) |
| (S)-2-((R)-2-aminopropanamido)-2-phenylethyl 2-isopropyl-2,3-dimethylbutanoate hydrochloride | (8b) |
| N-((S)-2-((R)-2-aminopropanamido)-2-phenylethyl)-2-isopropyl-2,3-dimethylbutanamide | (9a) |
| (S)-N-(2-amino-2-phenylethyl)-2-isopropyl-2,3-dimethylbutanamide hydrochloride | (9b) |
| (S)-2-(2-isopropyl-2,3-dimethylbutanamido)-1-phenylethyl glycinate hydrochloride | (10a) |
| (S)-2-((3S,5S,7S)-adamantane-1-carboxamido)-1-phenylethyl glycinate hydrochloride | (11a) |
| (3S,5S,7S)-N-((S)-2-hydroxy-2-phenylethyl) adamantane-1-carboxamide | (11b) |

TABLE 8
TRPM8 Activity

| Com- | | EC50 |
|---|---|---|
| pound | Structure | (μM) |
| (1a) | 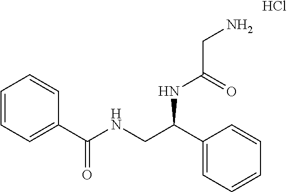 [SMILES: Cl ; NCC(=O)N[C@H](CNC(=O)c1ccccc1)c1ccccc1] | 0.655 |
| (1c) | 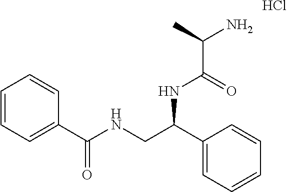 [SMILES: Cl ; C[C@@H](N)C(=O)N[C@H](CNC(=O)c1ccccc1)c1ccccc1] | 0.582 |
| (2c) | 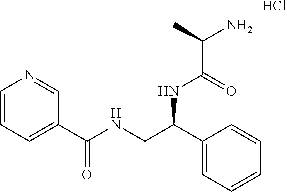 [SMILES: Cl ; C[C@@H](N)C(=O)N[C@H](CNC(=O)c1cccnc1)c1ccccc1] | 28 |
| (4a) | 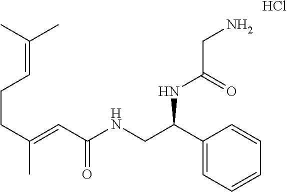 [SMILES: Cl ; CC(C)=CCC/C(C)=C/C(=O)NC[C@@H](NC(=O)CN)c1ccccc1] | 1.2 |
| (4b) | 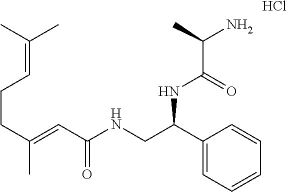 [SMILES: Cl ; CC(C)=CCC/C(C)=C/C(=O)NC[C@@H](NC(=O)[C@@H](C)N)c1ccccc1] | 1.45 |
| (6a) | 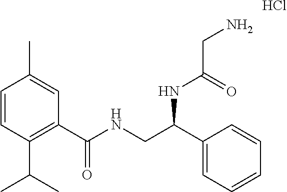 [SMILES: Cl ; Cc1ccc(C(C)C)c(C(=O)NC[C@@H](NC(=O)CN)c2ccccc2)c1] | 0.0019 |
| (6b) | 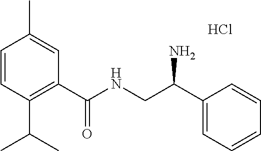 [SMILES: Cc1ccc(C(C)C)c(C(=O)NC[C@@H](N)c2ccccc2)c1 ; Cl] | 0.155 |
| (6c) | 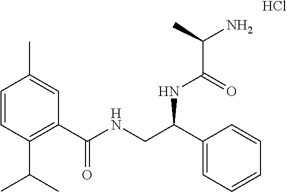 [SMILES: Cl ; Cc1ccc(C(C)C)c(C(=O)NC[C@@H](NC(=O)[C@@H](C)N)c2ccccc2)c1] | 0.0032 |
| (6d) | 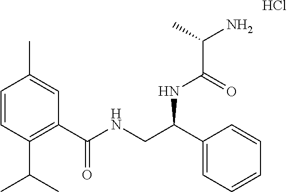 [SMILES: Cl ; Cc1ccc(C(C)C)c(C(=O)NC[C@@H](NC(=O)[C@H](C)N)c2ccccc2)c1] | 0.0066 |
| (7a) | 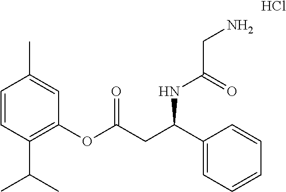 [SMILES: Cl ; Cc1ccc(C(C)C)c(OC(=O)C[C@@H](NC(=O)CN)c2ccccc2)c1] | 0.23 |
| (7b) | 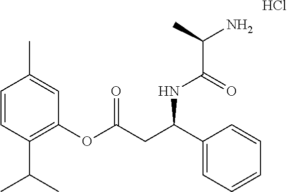 [SMILES: Cl ; Cc1ccc(C(C)C)c(OC(=O)C[C@@H](NC(=O)[C@@H](C)N)c2ccccc2)c1] | 0.060 |
| (8a) | 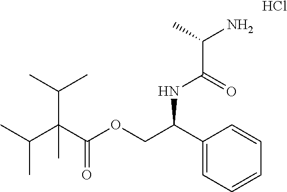 [SMILES: Cl ; CC(C)C(C)(C(=O)OC[C@@H](NC(=O)[C@H](C)N)c1ccccc1)C(C)C] | 0.415 |
| (8b) | 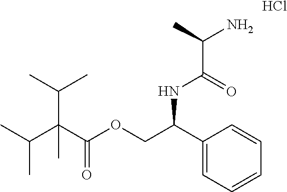 [SMILES: Cl ; CC(C)C(C)(C(=O)OC[C@@H](NC(=O)[C@@H](C)N)c1ccccc1)C(C)C] | 0.057 |
| (10a) | 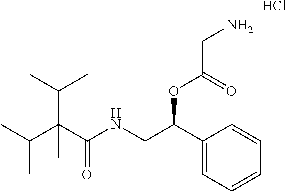 [SMILES: Cl ; CC(C)C(C)(C(=O)NC[C@@H](OC(=O)CN)c1ccccc1)C(C)C] | 0.00785 |
| (11a) | 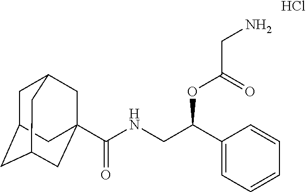 [SMILES: Cl ; NCC(=O)O[C@H](CNC(=O)C12CC3CC(CC(C3)C1)C2)c1ccccc1] | 0.01717 |
| Comp 1 | 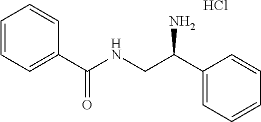 [SMILES: N[C@H](CNC(=O)c1ccccc1)c1ccccc1 ; Cl] | >10 |
| Comp 2 | 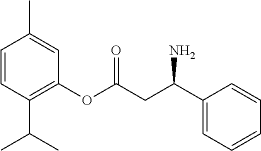 [SMILES: Cc1ccc(C(C)C)c(OC(=O)C[C@@H](N)c2ccccc2)c1] | 12 |
| Comp 3 | 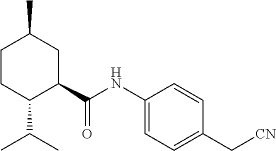 [SMILES: CC(C)[C@@H]1CC[C@@H](C)C[C@H]1C(=O)Nc1ccc(CC#N)cc1] | 0.60 |
| Comp 4 | 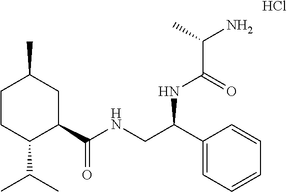 [SMILES: Cl ; CC(C)[C@@H]1CC[C@@H](C)C[C@H]1C(=O)NC[C@@H](NC(=O)[C@H](C)N)c1ccccc1] | 0.009 |

TRPM8 activation was determined by measuring intracellular calcium ion (Ca²⁺) level from transfected cells with the TRPM8 receptor gene, as described in EXAMPLE 12, the results of which are shown in TABLE 8. EC₅₀ values are provided in column 3 of TABLE 8, which measured the concentration of activating compound needed to reduce intracellular [Ca²⁺] by 50%. A lower intracellular [Ca²⁺] indicated TRPM8 was activated.

A compound was determined to be suitable for use as an activating compound if its EC₅₀ value was less than about 1 μM. Alternatively, a compound was determined to be suitable for use as an activating compound if its EC₅₀ value was less than about 0.6 μM. Alternatively, a compound was suitable for use an activating compound if its EC₅₀ vale was about the same as the EC₅₀ value of Comp 3 or less. (S)-2-((2-benzamido-1-phenylethyl)amino)-2-oxoethan-1-aminium chloride (1a), (R)-1-(((S)-2-benzamido-1-phenylethyl)amino)-1-oxopropan-2-aminium chloride (1c), (S)-2-((2-(2-isopropyl-5-methylbenzamido)-1-phenylethyl)amino-2-oxoethan-1-aminium chloride (6a), (S)-2-(2-isopropyl-5-methylbenzamido)-1-phenylethan-1-aminium chloride (6b), (R)-1-(((S)-2-(2-isopropyl-5-methylbenzamido)-1-phenylethyl)amino)-1-oxopropan-2-aminium chloride (6c), (S)-1-(((S)-2-(2-isopropyl-5-methylbenzamido)-1-phenylethyl)amino)-1-oxopropan-2-aminium chloride (6d), (R)-2-((3-(2-isopropyl-5-methylphenoxy)-3-oxo-1-phenylpropyl)amino-2-oxoethan-1-aminium chloride (7a), (R)-1-(((R)-3-(2-isopropyl-5-methylphenoxy)-3-oxo-1-phenylpropyl)amino)-1-oxopropan-2-aminium chloride (7b), (S)-2-((S)-2-aminopropanamido)-2-phenylethyl 2-isopropyl-2,3-dimethylbutanoate hydrochloride (8a), (S)-2-((R)-2-aminopropanamido)-2-phenylethyl 2-isopropyl-2,3-dimethylbutanoate hydrochloride (8b), (S)-2-(2-isopropyl-2,3-dimethylbutanamido)-1-phenylethyl glycinate hydrochloride (10a), and (S)-2-((3S,5S,7S)-adamantane-1-carboxamido)-1-phenylethyl glycinate hydrochloride (11a) all have EC₅₀ values that are less than about 1 μM, 0.6 μM, or the EC₅₀ value of Comp 3. Furthermore, Compounds 6a, 6c, 6d, and 10a had EC₅₀ values that were about the same or less than the EC₅₀ value of Comp 4.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound comprising the following structure or salts thereof: 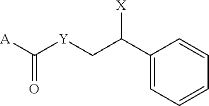
A is phenyl, pyridinyl, adamantyl, B, alkylsubstituted phenyl, alkylsubstituted pyridinyl, —O-phenyl, —O-pyridinyl, —O-adamantyl, —O—B, —O-(alkylsubstituted phenyl), —O-(alkylsubstituted pyridinyl), —N— phenyl, —N-pyridinyl, —N-adamantyl, —N—B, —N-(alkylsubstituted phenyl), or-N-(alkylsubstituted pyridinyl);
Y is —O—, —NH—, or nil in the case wherein A connects to the carbonyl functional group with an oxygen or nitrogen atom;
X is —OH, —O-AA, —NH₂, or —NH-AA;
B is tert-butyl, isopropyl, —C(isopropyl)₂(CH₃), or —(CH)═C(CH₃)—CH₂—CH₂—(CH)═C(CH₃)₂; and
AA is an amino acid.

2. The compound of claim 1, wherein the compound activates TRPM8.

3. The compound of claim 1, wherein A is phenyl, adamantyl, B, alkylsubstituted phenyl, 2-isopropyl-5-methylphenyl, —O-phenyl, —O-adamantyl, —O—B, —O-(alkylsubstituted phenyl), or —O-(2-isopropyl-5-methylphenyl).

4. The compound of claim 1, wherein the amino acid is alanine or glycine.

5. The compound of claim 3, wherein B is —C(isopropyl)₂(CH₃).

6. The compound of claim 1, wherein the compound has an EC₅₀ of less than about 1 μM.

7. The compound of claim 1, wherein the compound has an EC₅₀ of less than about 0.655 μM.

8. The compound of claim 1, wherein the compound has an EC₅₀ of less than about 0.6 μM.

9. The compound of claim 1, wherein the compound has an EC₅₀ of less than about an EC₅₀ value of G-180.

10. The compound of claim 1, wherein the compound is selected from the group consisting of (S)-2-((2-benzamido-1-phenylethyl)amino)-2-oxoethan-1-aminium chloride (1a), (R)-1-(((S)-2-benzamido-1-phenylethyl)amino)-1-oxopropan-2-aminium chloride (1c), (S)-2-((2-(2-isopropyl-5-methylbenzamido)-1-phenylethyl)amino-2-oxoethan-1-aminium chloride (6a), (S)-2-(2-isopropyl-5-methylbenzamido)-1-phenylethan-1-aminium chloride (6b), (R)-1-(((S)-2-(2-isopropyl-5-methylbenzamido)-1-phenylethyl)amino)-1-oxopropan-2-aminium chloride (6c), (S)-1-(((S)-2-(2-isopropyl-5-methylbenzamido)-1-phenylethyl)amino)-1-oxopropan-2-aminium chloride (6d), (R)-2-((3-(2-isopropyl-5-methylphenoxy)-3-oxo-1-phenylpropyl)amino-2-oxoethan-1-aminium chloride (7a), (R)-1-(((R)-3-(2-isopropyl-5-methylphenoxy)-3-oxo-1-phenylpropyl)amino)-1-oxopropan-2-aminium chloride (7b), (S)-2-((S)-2-aminopropanamido)-2-phenylethyl 2-isopropyl-2,3-dimethylbutanoate hydrochloride (8a), (S)-2-((R)-2-aminopropanamido)-2-phenylethyl 2-isopropyl-2,3-dimethylbutanoate hydrochloride (8b), (S)-2-(2-isopropyl-2,3-dimethylbutanamido)-1-phenylethyl glycinate hydrochloride (10a), and (S)-2-((3S,5S,7S)-adamantane-1-carboxamido)-1-phenylethyl glycinate hydrochloride (11a).

11. A method of activating TRPM8 comprising contacting the composition of claim 1 with an oral cavity.

12. A personal care composition comprising an activating compound with the following structure or salts thereof: 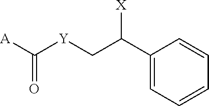
A is phenyl, pyridinyl, adamantyl, B, alkylsubstituted phenyl, alkylsubstituted pyridinyl, —O-phenyl, —O-pyridinyl, —O-adamantyl, —O—B, —O-(alkylsubstituted phenyl), —O-(alkylsubstituted pyridinyl), —N— phenyl, —N-pyridinyl, —N-adamantyl, —N—B, —N-(alkylsubstituted phenyl), or-N-(alkylsubstituted pyridinyl);
Y is —O—, —NH—, or nil in the case wherein A connects to the carbonyl functional group with an oxygen or nitrogen atom;
X is —OH, —O-AA, —NH₂, or —NH-AA;
B is tert-butyl, isopropyl, —C(isopropyl)₂(CH₃), or —(CH)═C(CH₃)—CH₂—CH₂—(CH)═C(CH₃)₂; and
AA is an amino acid.

13. The personal care composition of claim 12, wherein the personal care composition is an oral care composition.

14. The personal care composition of claim 12, wherein the personal care composition is an oral care composition selected from the group consisting of a dentifrice, a mouthrinse, a floss, a gum, and a whitening strip.

15. The personal care composition of claim 12, wherein the composition activates TRPM8.

16. The personal care composition of claim 12, wherein the composition has an EC₅₀ of less than about 1 μM.

17. The personal care composition of claim 12, wherein the composition has an EC₅₀ of less than about 0.655 μM.

18. The personal care composition of claim 12, wherein the activating compound is selected from the group consisting of (S)-2-((2-benzamido-1-phenylethyl)amino)-2-oxoethan-1-aminium chloride (1a), (R)-1-(((S)-2-benzamido-1-phenylethyl)amino)-1-oxopropan-2-aminium chloride (1c), (S)-2-((2-(2-isopropyl-5-methylbenzamido)-1-phenylethyl)amino-2-oxoethan-1-aminium chloride (6a), (S)-2-(2-isopropyl-5-methylbenzamido)-1-phenylethan-1-aminium chloride (6b), (R)-1-(((S)-2-(2-isopropyl-5-methylbenzamido)-1-phenylethyl)amino)-1-oxopropan-2-aminium chloride (6c), (S)-1-(((S)-2-(2-isopropyl-5-methylbenzamido)-1-phenylethyl)amino)-1-oxopropan-2-aminium chloride (6d), (R)-2-((3-(2-isopropyl-5-methylphenoxy)-3-oxo-1-phenylpropyl)amino-2-oxoethan-1-aminium chloride (7a), (R)-1-(((R)-3-(2-isopropyl-5-methylphenoxy)-3-oxo-1-phenylpropyl)amino)-1-oxopropan-2-aminium chloride (7b), (S)-2-((S)-2-aminopropanamido)-2-phenylethyl 2-isopropyl-2,3-dimethylbutanoate hydrochloride (8a), (S)-2-((R)-2-aminopropanamido)-2-phenylethyl 2-isopropyl-2,3-dimethylbutanoate hydrochloride (8b), (S)-2-(2-isopropyl-2,3-dimethylbutanamido)-1-phenylethyl glycinate hydrochloride (10a), and (S)-2-((3S,5S,7S)-adamantane-1-carboxamido)-1-phenylethyl glycinate hydrochloride (11a).

19. The personal care composition of claim 18, wherein the composition further comprises a TRPM8 activator.

20. The personal care composition of claim 19, wherein the composition further comprises a compound selected from the group consisting of TRPV1 agonist, TRPV1 antagonist, TRPV1 desensitizer, TRPA1 agonist, TRPA1 antagonist, TRPA1 desensitizer, TRPM8 agonist, TRPM8 antagonist, TRPM8 desensitizer, and combinations thereof.

21. A method of promoting thermogenesis comprising contacting one or more adipocytes with an activating compound, wherein the activating compound comprises the following structure or salts thereof: 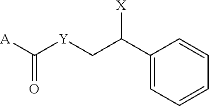
A is phenyl, pyridinyl, adamantyl, B, alkylsubstituted phenyl, alkylsubstituted pyridinyl, —O-phenyl, —O-pyridinyl, —O-adamantyl, —O—B, —O-(alkylsubstituted phenyl), —O-(alkylsubstituted pyridinyl), —N— phenyl, —N-pyridinyl, —N-adamantyl, —N—B, —N-(alkylsubstituted phenyl), or-N-(alkylsubstituted pyridinyl);
Y is —O—, —NH—, or nil in the case wherein A connects to the carbonyl functional group with an oxygen or nitrogen atom;
X is —OH, —O-AA, —NH₂, or —NH-AA;
B is tert-butyl, isopropyl, —C(isopropyl)₂(CH₃), or —(CH)═C(CH₃)—CH₂—CH₂—(CH)═C(CH₃)₂; and
AA is an amino acid.

22. The method of claim 21, wherein the method further comprises the steps of:
expressing a mitochondrial protein; and
activating one or more adipocytes to induce thermogenesis.

23. The method of claim 22, wherein the mitochondrial protein is selected from the group consisting of Ucp1, Ucp2, and combinations thereof.

24. The method of claim 23, wherein the method further comprises activating a receptor upon contact of activating compound with one or more adipocytes.

25. The method of claim 24, wherein the receptor is selected from the group consisting of TRPM8, PPARGC1A, alpha adrenergic receptor, beta adrenergic receptor, and gamma adrenergic receptor.

26. The method of claim 21, wherein one or more adipocytes are present in an affected area.

27. The method of claim 26, wherein the affected area has an excess of adipose tissue.

28. The method of claim 27, wherein the adipose tissue is selected from the group consisting of brown adipocytes, white adipocytes, beige adipocytes, brite adipocytes, subcutaneous adipose tissue, pericardial adipose tissue, marrow adipose tissue, and combinations thereof.

29. The method of claim 21, wherein an individual is treated by contacting the activating compound with one or more adipocytes.

30. The method of claim 29, wherein the treatment is selected from the group consisting of the treatment of obesity, the reduction of adipose tissue, body contouring and body shaping.

31. The method of use of claim 29, wherein the treatment is selected from the group consisting of type 1 diabetes, type 2 diabetes, insulin-resistance, dyslipidemia, irritable bowel syndrome, chronic pain, neuropathic pain, and inflammatory pain.

32. The method of claim 29, wherein the activating compound is contacted with one or more adipocytes through a route selected from the group consisting of injection, buccal, enteral, inhalable, infused, intramuscular, intrathecal, intravenous, nasal, ophthalmic, oral, otic, rectal, subcutaneous, sublingual, topical, transdermal, and combinations thereof.
